(19) United States
(12) Patent Application Publication
(10) Pub. No.: US 2015/0247205 A1
(43) Pub. Date: Sep. 3, 2015

(54) DIAGNOSIS OF MULTIPLE MYELOMA AND LYMPHOMA

(71) Applicants: The General Hospital Corporation, Boston, MA (US); Affymetrix, Inc., Santa Clara, CA (US)

(72) Inventors: Manoj Gandhi, Dublin, CA (US); Quan Nguyen, San Ramon, CA (US); David Tsai Ting, Dover, MA (US); Miguel Rivera, Cambridge, MA (US); Vikram Deshpande, Belmont, MA (US)

(21) Appl. No.: 14/634,108

(22) Filed: Feb. 27, 2015

Related U.S. Application Data
(60) Provisional application No. 62/080,580, filed on Nov. 17, 2014.
(60) Provisional application No. 61/946,468, filed on Feb. 28, 2014.

(51) Int. Cl. C12Q 1/68

(52) U.S. Cl. CPC C12Q 1/6886; C12Q 2600/16; C12Q 2600/158; C12Q 2600/118

(57) ABSTRACT

The present application relates to methods for diagnosing and treating multiple myeloma (MM) and non-Hodgkin lymphoma (NHL), e.g., based on the detection of clonal IgK or IgL-expressing cells.

### CLAIM OF PRIORITY

This application claims the benefit U.S. Provisional Patent Application Ser. Nos. 61/946,468, filed on Feb. 28, 2014, and 62/080,580, filed on Nov. 17, 2014. The entire contents of the foregoing are hereby incorporated by reference.

### TECHNICAL FIELD

The present application relates to methods for diagnosing and treating multiple myeloma (MM), Hodgkin lymphoma (HL), and non-Hodgkin lymphoma (NHL), and for differentiating MM and NHL from non-neoplastic lymph nodes, e.g., based on the detection of clonal IgK or IgL-expressing cells.

### BACKGROUND

Although relatively common, accounting for about 10% of new cancer diagnoses in the developed world (Howell et al., BMC Hematology 2013, 13:9), diagnosis of hematological malignancies can be challenging. Among the hematological malignancies are more than sixty different subtypes with differing clinical pathways and outcomes (Howell et al., supra; and Swerdlow et al., WHO classification of tumours of haematopoietic and lymphoid tissues, Fourth Edition. France: International Agency for Research on Cancer; 2008; Vardiman et al., Blood 2009, 114(5):937-951). Subtype-specific diagnosis of these blood cancers is critical for proper and safe treatments of patients as well as for preventing unnecessary medical expenses.

### SUMMARY

The present invention is based, at least in part, on the development of methods for accurately diagnosing and optionally treating MM, HL and NHL, e.g., based on detecting clonality of IgK/IgL.

Thus, provided herein are methods for diagnosing multiple myeloma (MM), Hodgkin lymphoma (HL), or non-Hodgkin lymphoma (NHL) in a subject. The methods include contacting a sample comprising cells from the subject with one or more polynucleotide probes that bind specifically to IgL mRNA in situ, and one or more polynucleotide probes that bind specifically to IgK mRNA in situ; detecting binding of the probes to IgL mRNA and IgK mRNA in cells in the sample, to determine numbers of IgL-expressing cells and IgK-expressing cells; calculating a ratio of IgL-expressing cells to IgK-expressing cells; identifying the IgK-expressing cells and IgL-expressing cells as plasma cells or B-lymphocytes, and:

(i) identifying a sample in which the ratio of IgL-expressing plasma cells to IgK-expressing plasma cells, or ratio of IgK-expressing plasma cells to IgL-expressing plasma cells, is above a threshold as being associated with MM;
(ii) identifying a sample in which the ratio of IgL-expressing B-lymphocytes to IgK-expressing B-lymphocytes, or ratio of IgK-expressing B-lymphocytes to IgL-expressing B-lymphocytes, is above a threshold as being associated with NHL;
or
(iii) identifying a sample in which the ratio of IgL-expressing cells to IgK-expressing cells, or ratio of IgK-expressing cells to IgL-expressing cells, is below a threshold as not being associated with MM or NHL. In some embodiments, the methods can include diagnosing Hodgkin lymphoma (HL), by (iv) identifying a sample with a mixture of light chain expressing and non-light chain expressing cells (i.e., a non-clonal); with IgK and IgL expression present in the cytoplasm; and the presence of characteristic Reed Sternberg (RS) cells, as being associated with HL.

Also provided herein is a method for diagnosing Hodgkin lymphoma (HL) in a subject. The method includes contacting a sample comprising cells from the subject with one or more polynucleotide probes that bind specifically to IgL mRNA in situ, and one or more polynucleotide probes that bind specifically to IgK mRNA in situ; detecting binding of the probes to IgL mRNA and IgK mRNA in cells in the sample; and identifying a sample with a non-clonal mixture of light chain expressing and non-light chain expressing cells, with IgK and IgL expression present in the cytoplasm and the presence of characteristic Reed Sternberg (RS) cells as being associated with HL.

Also provided herein are methods for selecting a treatment for a subject suspected of having multiple myeloma (MM), Hodgkin lymphoma (HL), or non-Hodgkin lymphoma (NHL). The methods include contacting a sample comprising cells from the subject with one or more polynucleotide probes that bind specifically to IgL mRNA in situ, and one or more polynucleotide probes that bind specifically to IgK mRNA in situ; detecting binding of the probes to IgL mRNA and IgK mRNA in cells in the sample, to determine numbers of IgL-expressing cells and IgK-expressing cells; calculating a ratio of IgL-expressing cells to IgK-expressing cells; identifying the IgK-expressing cells and IgL-expressing cells as plasma cells or B-lymphocytes, and:

(i) identifying a sample in which the ratio of IgL-expressing plasma cells to IgK-expressing plasma cells, or ratio of IgK-expressing plasma cells to IgL-expressing plasma cells, is above a threshold as being associated with MM, and selecting a treatment for MM for the subject;
(ii) identifying a sample in which the ratio of IgL-expressing B-lymphocytes to IgK-expressing B-lymphocytes, or ratio of IgK-expressing B-lymphocytes to IgL-expressing B-lymphocytes, is above a threshold as being associated with NHL, and selecting a treatment for NHL for the subject;
or
(iii) identifying a sample in which the ratio of IgL-expressing cells to IgK-expressing cells, or ratio of IgK-expressing cells to IgL-expressing cells, is below a threshold as not being associated with MM or NHL, and optionally not treating the subject. In some embodiments, the methods can include selecting a treatment for a subject suspected of having Hodgkin lymphoma (HL), by (iv) identifying a sample with a non-clonal mixture of light chain expressing and non-light chain expressing cells; with IgK and IgL expression present in the cytoplasm; and the presence of characteristic Reed Sternberg (RS) cells as being associated with HL, and selecting a treatment for HL for the subject.

Also provided herein is a method for selecting a treatment for a subject suspected of having Hodgkin lymphoma (HL). The method includes contacting a sample comprising cells from the subject with one or more polynucleotide probes that bind specifically to IgL mRNA in situ, and one or more polynucleotide probes that bind specifically to IgK mRNA in situ; detecting binding of the probes to IgL mRNA and IgK mRNA in cells in the sample; and identifying a sample with a non-clonal mixture of light chain expressing and non-light chain expressing cells, with IgK and IgL expression present in the cytoplasm and the presence of characteristic Reed Sternberg (RS) cells as being associated with HL, and selecting a treatment for HL for the subject.

Also provided herein are methods for treating a subject suspected of having multiple myeloma (MM), Hodgkin lymphoma (HL), or non-Hodgkin lymphoma (NHL). The methods include contacting a sample comprising cells from the subject with one or more polynucleotide probes that bind specifically to IgL mRNA in situ, and one or more polynucleotide probes that bind specifically to IgK mRNA in situ; detecting binding of the probes to IgL mRNA and IgK mRNA in cells in the sample, to determine numbers of IgL-expressing cells and IgK-expressing cells; calculating a ratio of IgL-expressing cells to IgK-expressing cells;

identifying the IgK-expressing cells and IgL-expressing cells as plasma cells or B-lymphocytes, and:
(i) identifying a sample in which the ratio of IgL-expressing plasma cells to IgK-expressing plasma cells, or ratio of IgK-expressing plasma cells to IgL-expressing plasma cells, is above a threshold as being associated with MM, and administering a treatment for MM to the subject;
(ii) identifying a sample in which the ratio of IgL-expressing B-lymphocytes to IgK-expressing B-lymphocytes, or ratio of IgK-expressing B-lymphocytes to IgL-expressing B-lymphocytes, is above a threshold as being associated with NHL, and administering a treatment for NHL to the subject;
or
(iii) identifying a sample in which the ratio of IgL-expressing cells to IgK-expressing cells, or ratio of IgK-expressing cells to IgL-expressing cells, is below a threshold as not being associated with MM or NHL, and optionally not treating the subject. In some embodiments, the methods can include treating a subject suspected of having Hodgkin lymphoma (HL), by
(iv) identifying a sample with a non-clonal mixture of light chain expressing and non-light chain expressing cells; with IgK and IgL expression present in the cytoplasm; and the presence of characteristic Reed Sternberg (RS) cells as being associated with HL, and administering a treatment for HL to the subject.

Also provided herein is a method for treating a subject suspected of having Hodgkin lymphoma (HL). The method includes contacting a sample comprising cells from the subject with one or more polynucleotide probes that bind specifically to IgL mRNA in situ, and one or more polynucleotide probes that bind specifically to IgK mRNA in situ; detecting binding of the probes to IgL mRNA and IgK mRNA in cells in the sample; and identifying a sample with a mixture of light chain expressing and non-light chain expressing cells, with IgK and IgL expression present in the cytoplasm and the presence of characteristic Reed Sternberg (RS) cells as being associated with HL, and administering a treatment for HL to the subject.

Further provided herein are methods for making a differential diagnosis between multiple myeloma (MM) and non-Hodgkin lymphoma (NHL) in a subject. The methods include contacting a sample comprising cells from the subject with one or more polynucleotide probes that bind specifically to IgL mRNA in situ, and one or more polynucleotide probes that bind specifically to IgK mRNA in situ; detecting binding of the probes to IgL mRNA and IgK mRNA in cells in the sample, to determine numbers of IgL-expressing cells and IgK-expressing cells; calculating a ratio of IgL-expressing cells to IgK-expressing cells; identifying the IgK-expressing cells and IgL-expressing cells as plasma cells or B-lymphocytes, and:

(i) diagnosing a subject in which the ratio of IgL-expressing plasma cells to IgK-expressing plasma cells, or ratio of IgK-expressing plasma cells to IgL-expressing plasma cells, is above a threshold as having MM; or
(ii) diagnosing a subject in which the ratio of IgL-expressing B-lymphocytes to IgK-expressing B-lymphocytes, or ratio of IgK-expressing B-lymphocytes to IgL-expressing B-lymphocytes, is above a threshold as having NHL.

In some embodiments, the methods can include making a differential diagnosis between MM, NHL, and Hodgkin lymphoma (HL). The methods include contacting a sample comprising cells from the subject with one or more polynucleotide probes that bind specifically to IgL mRNA in situ, and one or more polynucleotide probes that bind specifically to IgK mRNA in situ; detecting binding of the probes to IgL mRNA and IgK mRNA in cells in the sample, to determine numbers of IgL-expressing cells and IgK-expressing cells; calculating a ratio of IgL-expressing cells to IgK-expressing cells; identifying the IgK-expressing cells and IgL-expressing cells as plasma cells or B-lymphocytes, and:

(i) diagnosing a subject in which the ratio of IgL-expressing plasma cells to IgK-expressing plasma cells, or ratio of IgK-expressing plasma cells to IgL-expressing plasma cells, is above a threshold as having MM; or
(ii) diagnosing a subject in which the ratio of IgL-expressing B-lymphocytes to IgK-expressing B-lymphocytes, or ratio of IgK-expressing B-lymphocytes to IgL-expressing B-lymphocytes, is above a threshold as having NHL; or
(iii) diagnosing a subject in which a mixture of light chain expressing and non-light chain expressing cells with IgK and IgL expression present in the cytoplasm is present, and characteristic Reed Sternberg (RS) cells are present, as having HL.

In some embodiments of the methods described herein the threshold is 1.5:1, 2:1 or 3:1.

In some embodiments of the methods described herein, e.g., when a non-clonal mixture of cells is present, the step of identifying the IgK-expressing cells and IgL-expressing cells as plasma cells or B-lymphocytes can be omitted.

In some embodiments of the methods described herein, the sample is a biopsy sample obtained from the subject, and preferably wherein the sample comprises a plurality of individually identifiable cells. In some embodiments, the sample has been fixed, preferably with formalin, optionally embedded in a matrix, e.g., paraffin, e.g., a formaldehyde-fixed, paraffin-embedded (FFPE) clinical sample, and preferably wherein the sample has been sliced into sections.

In some embodiments of the methods described herein, (a) the one or more polynucleotide probes that bind specifically to IgL mRNA in situ, and the one or more polynucleotide probes that bind specifically to IgK mRNA in situ, are both applied to a single section from the sample, or (b) the one or more polynucleotide probes that bind specifically to IgL mRNA in situ, and the one or more polynucleotide probes that bind specifically to IgK mRNA in situ, are applied to consecutive sections from the sample. In some embodiments, the one or more polynucleotide probes that bind specifically to IgL mRNA in situ, and the one or more polynucleotide probes that bind specifically to IgK mRNA in situ, are both applied to a single section from the sample, and binding of the one or more polynucleotide probes to IgL is detected using a first detectable signal, and binding of the one or more polynucleotide probes to IgK is detected using a second detectable signal.

In some embodiments of the methods described herein, binding of the probes to IgL mRNA and IgK mRNA is detected using imaging, e.g., microscopy, e.g., bright-field or fluorescence microscopy, and preferably wherein at least three high power fields (HPF) (e.g., viewed using a 40× objective) in the mass are analyzed to determine the number of IgL-positive and IgK-positive cells.

In some embodiments, the methods described herein include detecting binding of the probes to IgL mRNA and IgK mRNA in the cytoplasm of the cells in the sample, to determine numbers of IgL-expressing cells and IgK-expressing cells.

In some embodiments of the methods described herein, the one or more probes comprise probes that bind to a plurality of target regions in the IgL or IgK mRNA.

In some embodiments of the methods described herein, the binding of the probes to IgL mRNA or IgK mRNA is detected using one or more labels that are directly or indirectly bound to the polynucleotide probes.

In some embodiments of the methods described herein, the binding of the probes to IgL mRNA or IgK mRNA is detected using branched nucleic acid signal amplification.

In some embodiments of the methods described herein, the probes are branched DNA probes.

In some embodiments, the methods described herein include contacting the sample with a plurality of probes that comprises one or more label extender probes that bind to one or more target regions in the IgL mRNA or IgK mRNA; hybridizing one or more pre-amplifier probes to the one or more label extender probes; hybridizing one or more amplifier probes to the pre-amplifier probes; and hybridizing one or more label probes to the one or more amplifier probes. In some embodiments, the label probes are conjugated to an enzyme, and binding of the probe is detected using a chromogen substrate with the enzyme. In some embodiments, the label probes are conjugated to a fluorophore, and binding of the probe is detected by observation of emissions from the fluorophore after illumination suitable to excite the fluorophore.

In some embodiments, the methods described herein include contacting a sample comprising tissue from the tumor with one or more polynucleotide probes that bind specifically to one or more mRNAs encoding a housekeeping gene (HKG) in situ; detecting binding of the one or more probes to one or more HKG mRNAs, and selecting for further analysis a sample in which binding of the one or more probes to the one or more HKG mRNAs are detected, or rejecting a sample in which binding of the one or more probes to the one or more HKG mRNAs are not detected. In some embodiments, the binding of the probes to IgL mRNA, IgK mRNA, or one or more HKG mRNAs is detected using branched nucleic acid signal amplification. In some embodiments, the probes are branched DNA probes.

In some embodiments, the methods described herein include contacting the sample with a plurality of probes that comprises one or more label extender probes that bind to a plurality of target regions in the IgL, IgK, or one or more HKG mRNAs; hybridizing one or more pre-amplifier probes to the one or more label extender probes; hybridizing one or more amplifier probes to the pre-amplifier; and hybridizing one or more label probes to the one or more amplifier probes.

In some embodiments of the methods described herein, the one or more polynucleotide probes that bind specifically to IgL mRNA in situ and the one or more polynucleotide probes that bind specifically to IgK mRNA in situ are applied to consecutive sections from the sample, the label probes are conjugated to an enzyme, binding of the IgL probes to IgL mRNA and IgK probes to IgK mRNA is detected using a first chromogen substrate for the enzyme, and binding of the HKG probes to the one or more HKG mRNAs is detected using a second chromogen substrate for the enzyme.

In some embodiments, the one or more polynucleotide probes that bind specifically to IgL mRNA in situ and the one or more polynucleotide probes that bind specifically to IgK mRNA in situ are applied to consecutive sections from the sample, the label probes are conjugated to a fluorophore, binding of the IgL probes to IgL mRNA and IgK probes to IgK mRNA is detected using a first fluorophore, and binding of the HKG probes to the one or more HKG mRNAs is detected using a second fluorophore.

In some embodiments, the one or more polynucleotide probes that bind specifically to IgL mRNA in situ and the one or more polynucleotide probes that bind specifically to IgK mRNA in situ are both applied to a single section from the sample, the label probes are conjugated to an enzyme, binding of the IgL probes to IgL mRNA is detected using a first chromogen substrate for the enzyme, binding of the IgK probes to IgK mRNA is detected using a second chromogen substrate for the enzyme, and binding of the HKG probes to the one or more HKG mRNAs is detected using a third chromogen substrate for the enzyme.

In some embodiments, the one or more polynucleotide probes that bind specifically to IgL mRNA in situ and the one or more polynucleotide probes that bind specifically to IgK mRNA in situ are both applied to a single section from the sample, the label probes are conjugated to a fluorophore, binding of the IgL probes to IgL mRNA is detected using a first fluorophore, binding of the IgK probes to IgK mRNA is detected using a second fluorophore, and binding of the HKG probes to the one or more HKG mRNAs is detected using a third fluorophore.

In some embodiments of the methods described herein, the cells of the sample were removed, at least in part, from a lymph node. In some embodiments, a sample identified as not being associated with MM or NHL is classified as being from a normal lymph node or a reactive lymph node based on one or more morphological features.

In some embodiments, classification of a normal lymph node is made, at least in part, based on a moderate expression of IgK/IgL within non-clonal lymphocytes of the lymphoid follicles. For example, moderate expression of IgK/IgL can be indicated by detection of up to 20 IgK/IgL mRNAs per lymphocyte.

In some embodiments, classification of a normal lymph node is made, at least in part, based on high expression of IgK/IgL within non-clonal plasma cells, e.g., high expression of IgK/IgL indicated by detection of 100 or more IgK/IgL mRNAs per plasma cell.

In some embodiments, classification of a reactive lymph node is made, at least in part, based on greater than a threshold number of lymphoid follicles showing a non-clonal population of IgK/IgL expressing lymphocytes; an exemplary threshold is 70% of the lymphoid follicles.

In some embodiments, classification of a reactive lymph node is made, at least in part, based on less than a threshold number of the lymphoid follicles showing a clonal population of IgK/IgL expressing lymphocytes; an exemplary threshold is 30% of the lymphoid follicles.

In some embodiments, classification of a reactive lymph node is made, at least in part, based on greater than a threshold number of non-clonal plasma cells per lymphoid follicle; e.g. a threshold of 3 non-clonal plasma cells per lymphoid follicle.

In some embodiments, classification of a reactive lymph node is made, at least in part, based on absence of clonal effacement within lymphoid follicles.

In some embodiments of the methods described herein, a sample identified as being associated with MM is identified, at least in part, based on one or more morphological features, e.g., based on high expression of IgK/IgL within a clonal population of plasma cells (e.g., wherein high expression of IgK/IgL is indicated by detection of 100 or more IgK/IgL mRNAs per plasma cell).

In some embodiments of the methods described herein, a sample identified as being associated with NHL is identified, at least in part, based on one or more morphological features, e.g., moderate expression of IgK/IgL within a clonal expansion of lymphocytes within lymphoid follicles (e.g., wherein moderate expression of IgK/IgL is indicated by detection of up to 20 IgK/IgL mRNAs per lymphocyte); more than half of the lymphoid follicles showing lymphocytes in which the ratio of IgL-expressing B-lymphocytes to IgK-expressing B-lymphocytes, or ratio of IgK-expressing B-lymphocytes to IgL-expressing B-lymphocytes, is above the threshold; presence of clonal effacement within lymphoid follicles; or less than a threshold number of plasma cells per lymphoid follicle (e.g., wherein the threshold is 7 plasma cells per lymphoid follicle).

The following definitions can be understood with reference to FIG. 1D. A “label extender” is a polynucleotide that is capable of hybridizing to both a nucleic acid analyte and also to at least a portion of a label probe system. A label extender typically has a first polynucleotide sequence L-1, which is complementary to a polynucleotide sequence of the nucleic acid analyte, and a second polynucleotide sequence L-2, which is complementary to a polynucleotide sequence of the label probe system (e.g., L-2 can be complementary to a polynucleotide sequence of a preamplifier, amplifier, a label probe, or the like). The label extender is preferably a single-stranded polynucleotide. Non-limiting examples of label extenders in various configurations and orientations are disclosed within, e.g., U.S. Published Patent Application No. 2012/0052498 (see, e.g., FIGS. 10A and 10B).

A “label probe system” comprises one or more polynucleotides that collectively comprise one or more label probes which are capable of hybridizing, directly or indirectly, to one or more label extenders in order to provide a detectable signal from the labels that are associated or become associated with the label probes. Indirect hybridization of the one or more label probes to the one or more label extenders can include the use of amplifiers, or the use of both amplifiers and preamplifiers, within a particular label probe system. Label probe systems can also include two or more layers of amplifiers and/or preamplifiers to increase the size of the overall label probe system and the total number of label probes (and therefore the total number of labels that will be used) within the label probe system. The configuration of the label probe system within a particular embodiment is typically designed in the context of the overall assay, including factors such as the amount of signal required for reliable detection of the target analyte in the assay, the particular label being used and its characteristics, the number of label probes needed to provide the desired level of sensitivity, maintaining the desired balance of specificity and sensitivity of the assay, and other factors known in the art.

An “amplifier” is a polynucleotide comprising one or more polynucleotide sequences A-1 and one more polynucleotide sequences A-2. The one or more polynucleotide sequences A-1 may or may not be identical to each other, and the one or more polynucleotide sequences A-2 may or may not be identical to each other. Within label probe systems utilizing amplifiers and label probes, polynucleotide sequence A-1 is typically complementary to polynucleotide sequence L-2 of the one or more label extenders, and polynucleotide sequence A-2 is typically complementary to polynucleotide sequence LP-1 of the label probes. Within label probe systems utilizing amplifiers, preamplifiers and label probes, polynucleotide sequence A-1 is typically complementary to polynucleotide sequence P-2 of the one or more preamplifiers, and polynucleotide sequence A-2 is typically complementary to polynucleotide sequence LP-1 of the label probes. Amplifiers can be, e.g., linear or branched polynucleotides.

A “preamplifier” is a polynucleotide comprising one or more polynucleotide sequences P-1 and one or more polynucleotide sequences P-2. The one or more polynucleotide sequences P-1 may or may not be identical to each other, and the one or more polynucleotide sequences P-2 may or may not be identical to each other. When one or more preamplifiers are utilized within a label probe system, polynucleotide sequence P-1 is typically complementary to polynucleotide sequence L-2 of the label extenders, and polynucleotide sequence P-2 is typically complementary to polynucleotide sequence A-1 of the one or more amplifiers. Preamplifiers can be, e.g., linear or branched polynucleotides.

A “label probe” is a single-stranded polynucleotide that comprises a label (or optionally that is configured to bind, directly or indirectly, to a label) to directly or indirectly provide a detectable signal. The label probe typically comprises a polynucleotide sequence LP-1 that is complementary to a polynucleotide sequence within the label probe system, or alternatively to the one or more label extenders. For example, in different embodiments, label probes may hybridize to either an amplifier and/or preamplifier of the label probe system, while in other embodiments where neither an amplifier nor preamplifier is utilized, a label probe may hybridize directly to a label extender.

A “label” is a moiety that facilitates detection of a molecule. Common labels in the context of the present invention include fluorescent, luminescent, light-scattering, and/or colorimetric labels. Suitable labels include enzymes and fluorescent moieties, as well as radionuclides, substrates, cofactors, inhibitors, chemiluminescent moieties, magnetic particles, and the like. Patents teaching the use of such labels include U.S. Pat. Nos. 3,817,837; 3,850,752; 3,939,350; 3,996,345; 4,277,437; 4,275,149; and 4,366,241. Labels include the use of enzymes such as alkaline phosphatase that are conjugated to an polynucleotide probe for use with an appropriate enzymatic substrate, such as fast red or fast blue, which is described within U.S. Pat. Nos. 5,780,227 and 7,033,758. Alternative enzymatic labels are also possible, such as conjugation of horseradish peroxidase to polynucleotide probes for use with 3,3′-Diaminobenzidine (DAB). Many labels are commercially available and can be used in the context of the invention.

The term “polynucleotide” encompasses any physical string of monomer units that correspond to a string of nucleotides, including a polymer of nucleotides (e.g., a typical DNA or RNA polymer), peptide nucleic acids (PNAs), modified oligonucleotides (e.g., oligonucleotides comprising nucleotides that are not typical to biological RNA or DNA, such as 2′-O-methylated oligonucleotides), and the like. The nucleotides of the polynucleotide can be deoxyribonucleotides, ribonucleotides or nucleotide analogs, can be natural or non-natural (e.g., locked nucleic acids, isoG or isoC nucleotides), and can be unsubstituted, unmodified, substituted or modified. The nucleotides can be linked by phosphodiester bonds, or by phosphorothioate linkages, methylphosphonate linkages, boranophosphate linkages, or the like. Polynucleotides can additionally comprise non-nucleotide elements such as labels, quenchers, blocking groups, or the like. Polynucleotides can be, e.g., single-stranded, partially double-stranded or completely double-stranded.

The term “probe” refers to a non-analyte polynucleotide.

Two polynucleotides “hybridize” when they associate to form a stable duplex, e.g., under relevant assay conditions. Polynucleotides hybridize due to a variety of well characterized physicochemical forces, such as hydrogen bonding, solvent exclusion, base stacking and the like. An extensive guide to the hybridization of nucleic acids is found in Tijssen (1993) Laboratory Techniques in Biochemistry and Molecular Biology-Hybridization with Nucleic Acid Probes, part I chapter 2, “Overview of principles of hybridization and the strategy of nucleic acid probe assays” (Elsevier, New York).

The term “complementary” refers to a polynucleotide that forms a stable duplex with its complement sequence under relevant assay conditions. Typically, two polynucleotide sequences that are complementary to each other have mismatches at less than about 20% of the bases, at less than about 10% of the bases, preferably at less than about 5% of the bases, and more preferably have no mismatches.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

The patent or application file contains at least one drawing executed in color. Copies of this patent or patent application publication with color drawing(s) will be provided by the Office upon request and payment of the necessary fee.

FIGS. 1A-B: Schematic representations of exemplary 1-plex tissue assay using a bDNA platform.

FIG. 1C: Schematic representation of an exemplary 2-plex tissue assay using a bDNA platform.

FIG. 1D: Schematic illustration of an exemplary bDNA amplification scheme.

FIG. 2. A diagrammatic representation of a reactive lymph node showing lymphoid follicles. Reactive lymph nodes show germinal centers comprising of non-clonal activated B-lymphocytes and plasma cells surrounded by a mantle zone rim of non-clonal B-lymphocytes.

FIG. 3. A diagrammatic representation of a follicular lymphoma in a lymph node showing malignant lymphoid follicles. Malignant lymphoid follicles comprise a clonal population of B-lymphocytes and are surrounded by a mantle zone rim of non-clonal B-lymphocytes. The process of replacement of normal lymphoid follicular architecture with malignant lymphocytes is referred to as clonal effacement. There are very few plasma cells within the malignant follicle. The inter-follicular areas show presence of non-clonal plasma cells.

FIGS. 4A-B show exemplary ISH results from Normal/Reactive LN (IGKC-IGLC non-clonal), obtained using 1-plex RNA ISH (4A) or 2-plex RNA ISH (4B).

FIGS. 5A-B show ISH results from Multiple Myeloma samples obtained using 1-plex RNA ISH (5A: top, IgK clonal; bottom, IgL clonal) or 2-plex RNA ISH (5B, IgL clonal).

FIGS. 6A-B show exemplary ISH results from IgKC-clonal Non-Hodgkin Lymphoma, obtained using 1-plex RNA ISH (6A) or 2-plex RNA ISH (6B).

FIG. 7 shows exemplary ISH results from IgLC-clonal Non-Hodgkin Lymphoma, obtained using 1-plex RNA ISH.

FIGS. 8A-B. Schematic illustrations of exemplary algorithms for differential diagnosis of MM from NHL, and also a differential diagnosis of normal versus reactive lymph nodes for non-neoplastic samples, without (8A) or with (8B) detection of one or more housekeeping genes (HKG).

FIGS. 9A-B are exemplary algorithms that are especially useful in the case where the IGLC probe cross-reacts with other (nuclear) IGL-Like targets such as IGLL5. FIG. 9A is a schematic illustration of an exemplary algorithm for simultaneous diagnosis of Reactive LN, myeloma and lymphoma, and FIG. 9B is a schematic illustration of an exemplary interpretive algorithm for diagnosing Reactive LN, myeloma and lymphoma using IGKC/IGLC staining pattern. As shown in 9B, nuclear staining with the IGLC probe is disregarded.

### DETAILED DESCRIPTION

Described herein are methods for the simultaneous and accurate diagnosis of multiple myeloma, Hodgkin lymphoma, and non-Hodgkin lymphoma, using in situ hybridization to detect clonal cells expressing immunoglobulin (Ig) Kappa and/or Lambda light chain mRNA.

Lymphocytes and Plasma Cells

Lymphocytes are the main cell type of the immune system. There are three major types of lymphocytes: T cells, B cells, and natural killer (NK) cells. Lymphocytes typically have a large nucleus that can be used to distinguish them from other cells in the blood.

B lymphocytes are a type of white blood cells that originate in the bone marrow and have the ability to differentiate into specialized cells called plasma cells; this differentiation step typically occurs in the lymph nodes. Plasma cells are a source of Immunoglobulins (Ig). In mammals, the structure of an Ig includes two Ig heavy chains and two Ig light chains. There are two types of light chains, Kappa (K) and Lambda (L). K is encoded by a locus on chromosome 2p12 (GenBank Acc. No. NG_{—}000834.1), while L is encoded by a locus on chromosome 22q11.2 (GenBank Acc. No. NG_{—}000002.1).

Each lymphocyte or plasma cell produces only one class of light chain. Normal or reactive (enlarged due to antigen stimulus) lymph nodes have a mixed (non-clonal) population of K and L expressing lymphocyte and plasma cells in a ratio below a threshold for clonality. This threshold can vary depending on the sample being examined, and can be, e.g., about 2:1 in serum (measuring intact whole antibodies) or 1:1.5 if measuring free light chains. For clarity, a ratio of 2:1 for K:L means that of the cells in the sample at issue, ⅔ are expressing K and ⅓ are expressing L. Depending on factors such as manner of sample preparation, the characteristics of the assay at issue, the threshold for clonality can be adjusted through routine testing. For example, the 2:1 K:L ratio for clonality can be expanded to 3:1, or the 1:1.5 K:L ratio decreased to 7:4. As would be known by one of skill in the art, it is unlikely to have a non-clonal sample with a ratio higher than 3:1. It should be noted that with respect to these ratios, either K or L can be the dominant species (e.g., a ratio such as the 2:1 serum ratio can be either 2:1 for K:L or 2:1 for L:K). See, e.g., Katzmann et al., Clin Chem. 2002 September; 48(9):1437-44; Nelson et al., Br J Haematol 1990; 81:223-230; Bhole et al, Ann Clin Biochem Jan. 31, 2014 (Published online before print Jan. 31, 2014, doi: 10.1177/0004563213518758).

Multiple Myeloma (MM)

Multiple myeloma (MM) is a disease of white blood cells caused by malignant proliferation of plasma cells. Myeloma cells divide uncontrollably to form masses, typically at multiple sites within the bone marrow, which are comprised of neoplastic plasma cells expressing the same type of Ig light chain, either K or L; this phenomenon, in which greater than a threshold number of the plasma cells express one type of light chain, is called clonality, and as discussed above, this threshold can be, e.g., 1.5:1, 2:1 or 3:1. Thus, a sample in which the K:L ratio is, e.g., 8:1 or 9:1, would be classified as possessing clonality.

Since plasma cells show very high expression of K or L light chains, these cells can be detected within a molecular method for diagnosis of MM in tissue via immunohistochemistry (IHC)/antibody assays or RNA ISH assays targeting the constant fragment of Kappa (IgKC) or Lambda (IgLC) subtype of Ig.

However, RNA ISH diagnostic tests currently in clinical use detect IgKC or IgLC mRNA in plasma cells in a single-plex format. Because of this limitation, two successive tissue sections from the same tissue block are required in order to make the diagnosis of MM using this technique. Furthermore, if the expression of one or more housekeeping genes is to be assessed in these assays (e.g., to assess RNA integrity), even further sections must be taken from the tissue block, which may not be possible depending on the amount of tissue collected and that remains available for use, and adds a further complication through having different sample sections with potentially varying characteristics being used for the test. Single-plex RNA IHC and ISH assays are commercially available, such as the BenchMark® IHC/ISH instrument family and the corresponding Kappa and Lambda probes and antibodies (Ventana Medical Systems, Inc., Tucson, Ariz.).

Symptoms of MM include elevated calcium, renal failure, anemia, and bone lesions (International Myeloma Working Group, Br. J. Haematol. 121 (5): 749-57 (2003)).

Non-Hodgkin Lymphoma (NHL) Non-Hodgkin Lymphoma (NHL) is similar to MM except that neoplastic cells are derived from B-lymphocytes, which as noted above are the precursors of the plasma cells that are malignant in MM. Malignant B-lymphocytes in NHL also exhibit the phenomenon of IgKC/IgLC clonality. However, the expression level of IgK and IgL in B-lymphocytes is significantly lower than that in plasma cells, and thus cannot be detected by standard RNA ISH based techniques that are not sensitive enough to detect such low levels of expression.

Presently, a common method for the diagnosis of NHL is RT-PCR for detection of Ig chromosomal rearrangement (See, e.g., Stahlberg et al., “Quantitative real-time PCR method for detection of B-lymphocyte monoclonality by comparison of kappa and lambda immunoglobulin light chain expression”, Clin. Chem., 49(1):51-9 (2003); and Pott et al., “MRD Detection in B-Cell Non-Hodgkin Lymphomas Using Ig Gene Rearrangements and Chromosomal Translocations as Targets for Real-Time Quantitative PCR”, Lymphoma: Methods and Protocols, Methods in Molecular Biology, vol. 971, 175-200 (2013)). These methods require extraction of B-lymphocytes from tumor tissues, culturing of B-lymphocytes, DNA purification and RT-PCR. These methods have several disadvantages, including a lengthy wait time of 2-3 weeks to obtain results. In addition, these methods commonly require that at least 70% or greater of the tissue be tumor tissue to obtain a desired level of sensitivity. This can be challenging because the distinction of malignant lymphocytes from normal lymphocytes is very difficult, especially in the small tumor foci typically seen in early stages of lymphoma. Further, these methods involve homogenization of tissues, which destroys key morphological features that are critical in the interpretation and diagnosis of the disease. Finally, in the art-known methods, diagnostic assays for MM and NHL are performed separately, which requires more precious samples to be collected from the patient, increasing costs and requiring patients to wait for weeks for test outcomes.

Hodgkin Lymphoma (HL)

The World Health Organization has classified Hodgkin lymphoma into five types: nodular sclerosing, mixed cellularity, lymphocyte depleted, lymphocyte rich, and nodular lymphocyte-predominant (Jaffe et al., eds. World Health Organization Classification of Tumours: Pathology and Genetics of Tumours of Haematopoietic and Lymphoid Tissues. Lyon, France: IARC Press; 2001).

Presently, a diagnosis is made based on laboratory tests (e.g., Complete blood cell (CBC) count studies for anemia, lymphopenia, neutrophilia, or eosinophilia; elevated erythrocyte sedimentation rate (ESR), Lactate dehydrogenase (LDH), or serum creatinine) and imaging studies (including x-rays, computed tomography (CT) scans, and positron emission tomography (PET) scans), but a histologic diagnosis of Hodgkin lymphoma is always required (e.g., using an excisional lymph node biopsy sample) showing the presence of mononucleate and binucleate Reed-Sternberg cells. A bone marrow biopsy is sometimes indicated.

Symptoms can include asymptomatic lymphadenopathy, constitutional or “B” symptoms (e.g., unexplained weight loss, unexplained fever, night sweats); intermittent fever; chest pain, cough, shortness of breath, or a combination of those; pruritus; pain at sites of nodal disease, precipitated by drinking alcohol; or back or bone pain.

Methods of Detection and Diagnosis

The present methods can be used to make an accurate diagnosis of MM versus NHL, or MM versus NHL versus HL. Preferred embodiments include performing a semiquantitative ratiometric analysis of the proportion of IgK-expressing cells in comparison to IgL-expressing cells, and determining whether the cells are plasma cells or B-lymphocytes. An IgK/IgL ratio that significantly differs from normal (i.e., normal is non-clonal), such as a ratio that is over a set threshold, e.g., over 1.5:1, preferably over 2:1, more preferably over 3:1, confirms a diagnosis of clonality (MM or NHL) versus non-clonality (normal or reactive cells); with the identification of the monoclonal cells as plasma cells or B-lymphocytes being used, at least in part, in the identification of the cancer as MM (plasma cells), or NHL (B-lymphocytes). When K/L clonality is present, it is common that a very high percentage of the plasma cells or lymphocytes at issue will be expressing either K or L, such as at a ratio of 8:1 or 9:1 of K:L or L:K.

The identification of the cell type and IgK/IgL ratio are critical components in the diagnosis of MM and NHL.

To provide a quantitative IgK/IgL ratio, an in situ hybridization assay, preferably but not necessarily a branched nucleic acid (bDNA) signal amplification ISH assay, is used to estimate an IgK/IgL ratio. In some embodiments, an identification of plasma cells versus B-lymphocytes can be made using one or more of the criteria in Table A after RNA ISH staining for IgK/IgL:

TABLE A

| Plasma Cells | B-lymphocytes |
|---|---|
| Intense and dark cytoplasmic staining (e.g., | Cells do not have intense cytoplasmic |
| if a CISH approach is used with alkaline | staining, but instead have staining that is |
| phosphatase and fast red, an intense and dark | commonly at least 2x, preferably 4x, more |
| red color) that is commonly at least 2x, | preferably 6x and most preferably 10x |
| preferably 4x, more preferably 6x and most | weaker than the staining observed in plasma |
| preferably 10x stronger than the staining | cells |
| observed in B-lymphocytes | Cells show moderate expression of IgK/IgL, |
| Cells show high expression of IgK/IgL, | which generally results in, e.g., 2-20 dots |
| which generally results in, e.g., greater than | (e.g., the signal generated by the ISH assay |
| 100 dots, such that distinguishing individual | for the detection of IgK/IgL RNA |
| dots from one another is difficult, but with | molecules), with individual dots being more |
| the number of visible dots and their | easily distinguished from one another than in |
| distinctness dependent on the RNA ISH | plasma cells but with the number of visible |
| assay utilized | dots also dependent on the RNA ISH assay |
| Even staining at 4-40x magnification | utilized |
| | Granular staining at 10-40x magnification |

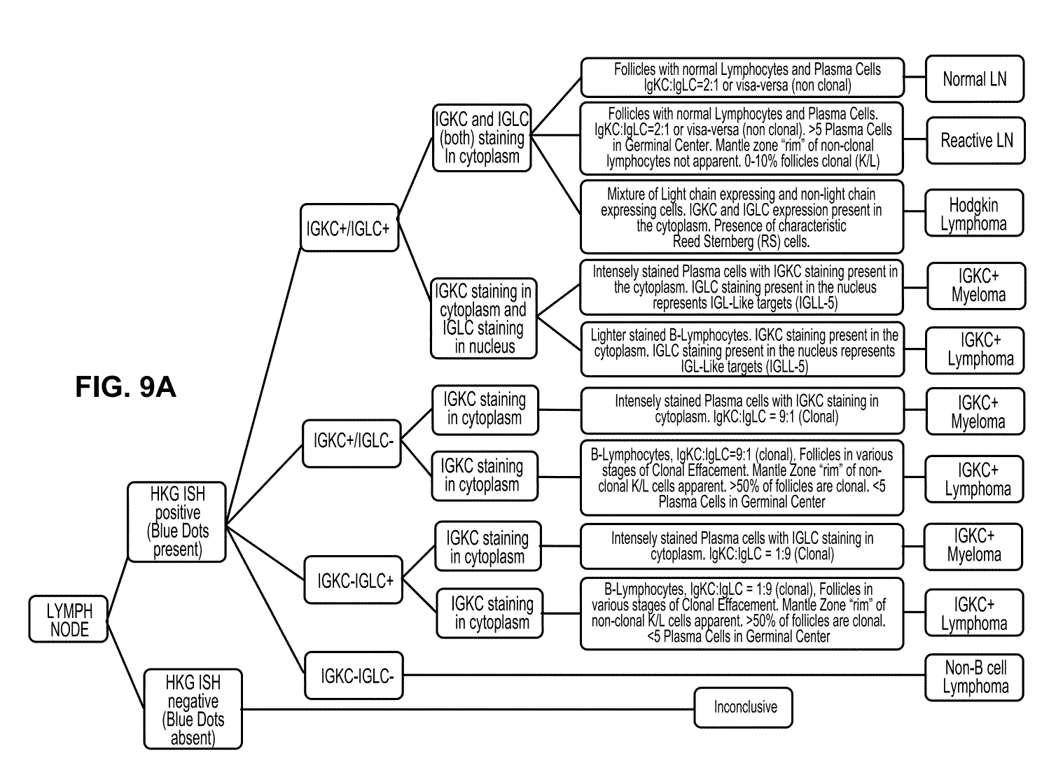

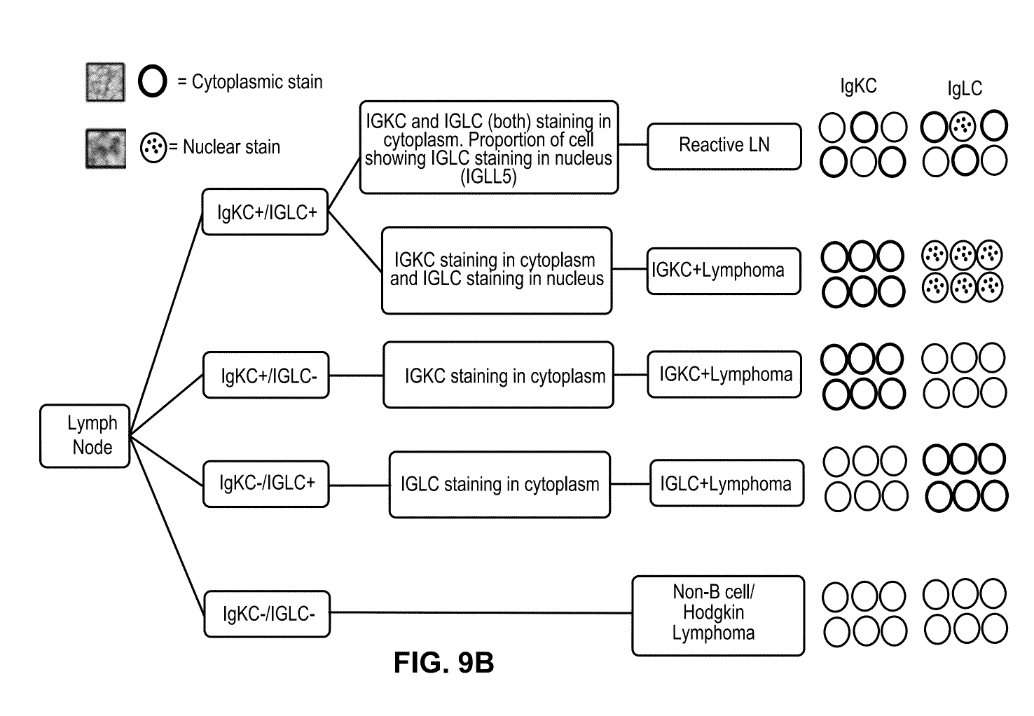

In some cases, the IGLC probe may show cross reactivity to IGL-Like targets (e.g., IGLL5). However, IGLC and IGL-Like staining can be readily distinguished based on the sub-cellular localization of the signals. IGLC staining is seen in the cytoplasm of cells (i.e., of B-Lymphocytes and plasma cells), while IGL-Like staining (e.g., IGLL5) is seen primarily in the nucleus of cells (e.g., of B-Lymphocytes and Plasma cells). Thus in some embodiments only cells with IGLC staining in the cytoplasm, and not in the nucleus, are considered to express IGLC (i.e., to be IGLC+) for the purposes of the present methods; cells with IGLC staining only in the nucleus are not considered to be IGLC+, but rather are considered to be IGKC+ cells (and if a 2 color assay was performed with 1st probe set and color for IGKC and a 2nd probe set and color for IGLC, then IGKC staining in the cytoplasm would have been observed); cells with IGLC staining in both the nucleus and cytoplasm (with it likely occurring primarily in the nucleus), are considered IGKC+ cell (and again, if a 2 color assay was performed, then IGKC staining in the cytoplasm would have been seen; any IGLC staining seen in the cytoplasm is presumably IGL-Like staining from IGLL5). Thus, in some embodiments the methods include disregarding any staining in the nucleus, e.g., disregarding IGLC staining in the nucleus. An exemplary algorithm for simultaneous diagnosis of reactive LN, myeloma and lymphoma for this situation is shown in FIG. 9A. An exemplary interpretive algorithm for this situation is shown in FIG. 9B.

Diagnosing MM, HL, or NHL—Identifying Clonal Cells in a Sample

The methods described herein detect RNA in situ, e.g., in formalin fixed paraffin embedded material, fresh frozen tissue sections, fine needle aspirate biopsies, or tissue microarrays. In many embodiments, the sample is taken from a lymph node, and commonly an enlarged lymph node, but many other tissue sources can also be assayed with the methods described herein. Other non-limiting tissues include soft tissue mass, mucosa-associated lymphoid tissue (MALT) from the gut, bone marrow, and blood. The methods described herein can also be performed in other RNA ISH contexts, such as with cellular samples, such as cells isolated from blood (including whole blood), bone marrow or sputum (such as samples prepared using centrifugation (such as with the CytoSpin Cytocentrifuge instrument (ThermoFisher Scientific, Waltham, Mass.) or smeared on a slide), blood smears on slides (including whole blood smears). In some embodiments, the methods are performed on cells from a mass, e.g., a mass suspected of being MM or NHL (e.g., from bone or bone marrow), and can be performed, e.g., either as described above or in alternative approaches such as a RNA ISH based flow cytometry setting.

In some embodiments of the present methods, the sample is analyzed using RNA ISH to determine the number of IgL-positive (IgL+) cells and IgK-positive (IgK+) cells, and the ratio of IgK+ to IgL+ cells is determined; and in preferred embodiments, the cell type of the IgK/IgL expressing cells is determined by the presence (in plasma cells) or absence (in B lymphocytes) of intense cytoplasmic staining (e.g., numerous dots such that individual dots are not discernible at 4-40× magnification), as determined with IgK and/or IgL probes. In many embodiments, it is beneficial if one or more of the following are excluded from the analysis: (1) staining outside of the cytoplasm of the lymphocyte and plasma cells (e.g., lumen, fatty tissue, muscle tissue), (2) nuclear staining within the cells, whether the cells are lymphocytes or plasma cells, particularly when it is possible to differentiate between the nucleus and the cytoplasm, and (3) lymphocytes or plasma cells that show less dots than would be expected for the background signal of the RNA ISH assay at question. For factor (3) in particular, the background signal and the number of dots at issue will vary depending on the particular assay, but can be routinely determined for a specific assay by one of skill in the art. For example, in certain bDNA ISH assays, lymphocytes that show less than 5, and preferably less than 2 or 3, dots in the cytoplasm of a particular lymphocyte should be disregarded for the analysis.

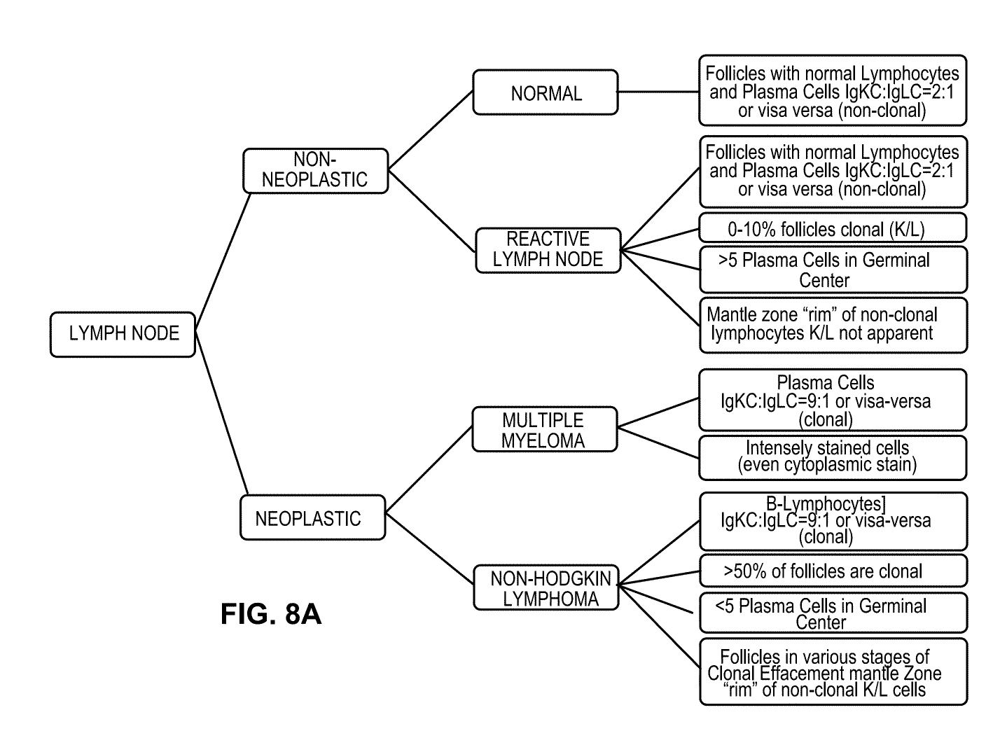

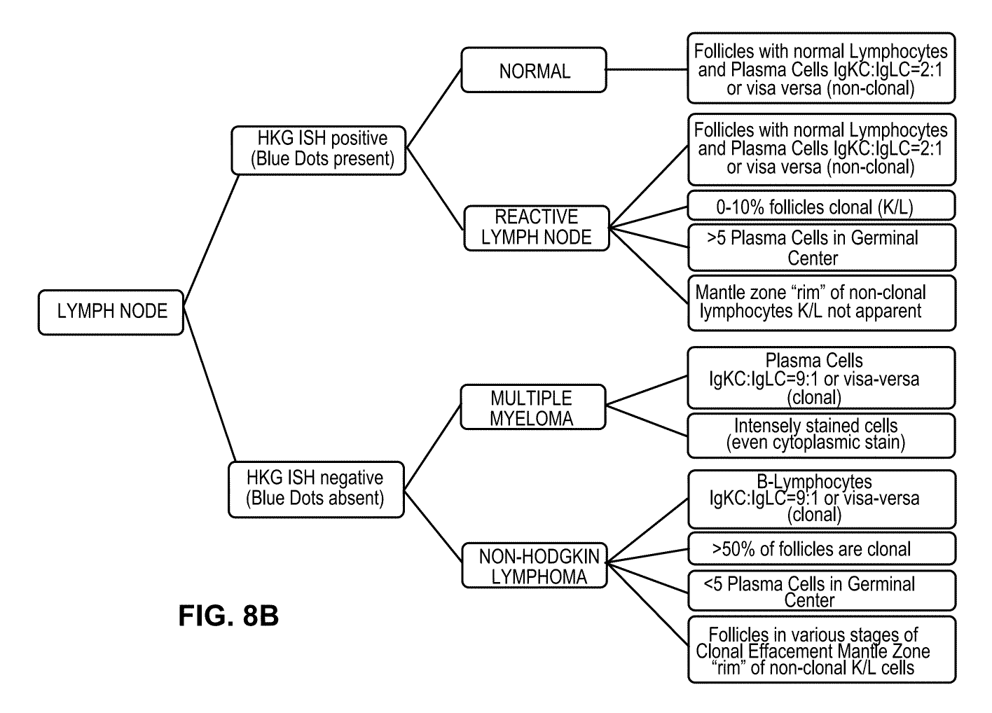

Once the numbers of IgL positive (IgL+) and IgK positive (IgK+) cells in a sample are determined, as shown in FIGS. 8A-B and 9A-9B, if the ratio of IgL+:IgK+ cells, or IgK+:IgL+ cells is over a set threshold, e.g., over 3:1, preferably over 4:1, more preferably over 5:1, or even more preferably over 6:1 or higher (such as 8:1 or 9:1), the sample is identified as likely being from a mass associated with MM or NHL. If the ratio of IgL+:IgK+ cells, or IgK+:IgL+ cells is below a threshold, e.g., is less than 3:1, the sample is identified as likely to be from a normal or reactive tissue. When a mixture of cells with IgK+ and IgL+ in the cytoplasm are present, as well as the characteristic Reed-Sternberg cells, the sample is identified as likely being from a mass associated with HL.

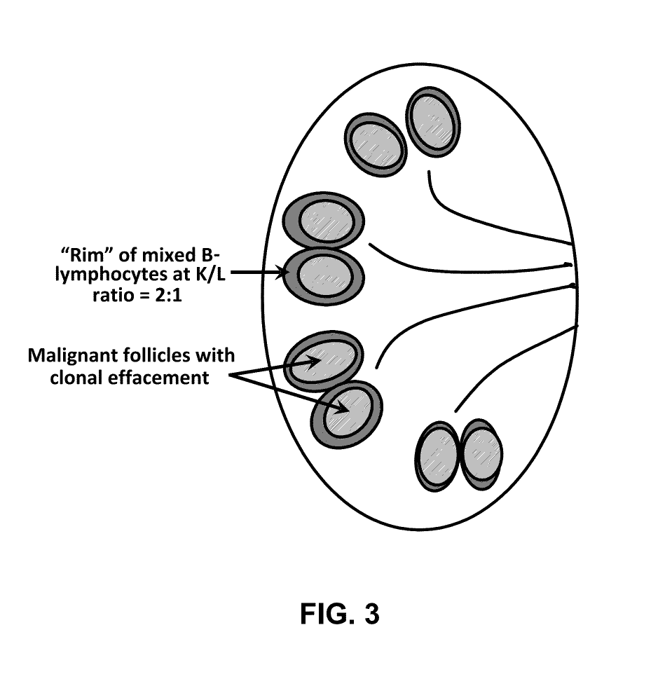

Morphological and other features that can be seen with ISH and not in other assay types, e.g., lysate assays such as RT-PCR, can be used to provide additional factors in identifying a sample. For example, when the sample comprises lymph node tissue and after a determination that a neoplasm is not at issue based on non-clonal expression of K/L, a further determination that a sample is from a normal lymph node and not a reactive lymph node can be made based on the presence of one or more of the following morphological features seen by ISH:
1. Lymphoid follicles with non-clonal population of light chain-expressing B-lymphocytes with moderate expression of K/L, e.g., 2-20 dots/cell. At this level of K/L expression, the staining can be, e.g., 2-10× less intense than the staining observed in plasma cells and their corresponding higher level of K/L expression. Additionally, it is easier to distinguish individual dots from one another at this level of expression, relative to distinguishing individual dots within plasma cells. As discussed earlier, the normal ratio of Kappa (IgKC) to Lambda (IgLC) expressing lymphocytes is around 2:1 IgKC:IgLC, or vice-versa, but can vary to a certain degree (e.g., the ratio can be 1.5:1). Additionally, a hematoxylin stain will produce a uniform dark-blue stain within the nuclei of the lymphocytes.
2. Presence of non-clonal population of light chain-expressing plasma cells that exhibit dark ISH-stain covering the entire cytoplasm of the cell (e.g., with greater than 100 dots per cells). At this level of K/L expression, the staining can be, e.g., 2-10× more intense than the staining observed in B-lymphocytes and their corresponding lower level of K/L expression. Additionally, it is more difficult to distinguish individual dots from another at this level of expression, relative to distinguishing individual dots within B-lymphocytes. The ratio of Kappa (IgKC) to Lambda (IgLC) expressing plasma cells is within a normal range as discussed earlier (e.g., 2:1, 1.5:1).
3. Few activated follicles that show the presence of germinal centers (e.g., 1 activated follicle with the rest not having germinal centers) (see reactive lymph node below).
   In contrast, a determination of a reactive lymph node can be made based on the presence of one or more of the following morphological features seen by ISH:
1. Lymphoid follicles with presence of germinal centers having non-clonal population light chain-expressing B-lymphocytes. The ratio of Kappa (IgKC) to Lambda (IgLC) expressing lymphocytes is within the normal range (e.g., 2:1 IgKC:IgLC, or vice-versa). Greater than a threshold number of lymphoid follicles consist of non-clonal B-lymphocytes, with this threshold being, e.g., greater than 70%, preferably greater than 80%, and more preferably greater than 90% of the B-lymphocytes are non-clonal.
2. Less than a threshold number of the lymphoid follicles may show clonal population of B-lymphocytes with predominance of one population of light-chain expression (IgKC or IgLC). This threshold is less than 30%, preferably less than 20% and more preferably less than 10%. This feature of reactive lymph nodes is seen particularly in children and in patients with immune deficiency.
3. Since the germinal centers consist of non-clonal population of B-lymphocytes too, the surrounding mantle-zone rim of non-clonal lymphocytes is not apparent when performing an RNA ISH assay. The mantle-zone rim, however, is still visible when doing an H&E stain.
4. Lymphoid follicles often show presence of non-clonal plasma cells per follicle above a threshold, e.g., more than 3, preferably more than 4, more preferably more than 5, and most preferably more than 6 non-clonal plasma cells per follicle).
5. Absence of clonal effacement (see Non-Hodgkin Lymphoma and FIG. 3), where lymphoid follicles are in various stages of replacement of normal non-clonal B-lymphocytes (e.g., where the K:L expression ratio is within a normal range such as 2:1 K:L, or vice-versa) with malignant clonal B-lymphocytes (e.g., where the K:L expression ratio is over a threshold such as 9:1 K:L, or vice-versa). Clonal effacement originates in the center of the follicle and progresses outwards to the periphery of the follicle.

A determination that a sample with mixed IgK and IgL cells (non-clonal) is from a NH mass can be made based on the presence of Reed-Sternberg cells, which are CD30 and CD15 positive, large, and either multinucleated or have a bilobed nucleus (e.g., detected using standard light microscopy methods). See, e.g., Kumar et al., Robbins Basic Pathology, Ninth Edition (Saunders 2012).

After a determination of clonality has been made, a further determination of Multiple Myeloma can be made based on one or more of the following morphological features seen by ISH:
1. Tumor comprising of a clonal population of plasma cells (as evidenced by, e.g., intense dark ISH stain for K/L covering the entire cytoplasmic area of the cells). At this level of K/L expression, it is more difficult to distinguish individual dots from one another within the cells.
2. A ratio of IgKC to IgLC expressing plasma cells above a threshold (e.g., 7:1, 8:1, 9:1 of IgKC:IgLC expressing cells, or vice-versa). The normal ratio of K/L expressing plasma cells in non-myeloma cases is e.g., 1.5:1, 2:1 K:L, or vice-versa.

Additionally or alternatively, the further determination of Multiple Myeloma can be made based on one or more of the following morphological features seen by ISH:
1. IGKC+ myeloma cells show strong positive cytoplasmic staining with IGKC probe and may also show staining with IGLC probe predominantly in the nucleus due to the presence of IGL-Like targets (e.g., IGLL5).
2. IGLC+ myeloma cells show strong positive cytoplasmic staining with IGLC probe and do not show staining with IGKC probe.
   After a determination of clonality, a further determination of Non-Hodgkin Lymphoma can be made based on one or more of the following morphological features seen by ISH:
1. Lymphoid follicles show clonal expansion of B-lymphocytes, and with moderately stained cells (e.g., with 2-20 K/L dots in the cytoplasm of the cells). At this level of K/L expression, the staining can be, e.g., 2-10× less intense than the staining observed in plasma cells and their corresponding higher level of K/L expression. Additionally, it is easier to distinguish individual dots from one another at this level of expression, relative to distinguishing individual dots within any plasma cells that may be present.
2. Majority of the follicles (>50%) show IgKC/IgLC expressing B-lymphocytes in a ratio above the normal threshold (e.g., at a ratio of 7:1, 8:1, 9:1 of IgKC/IgLC expressing cells, or vice-versa). The ratio of K/L expressing B-lymphocytes in normal or reactive lymph node is e.g., 1:5:1, 2:1 K:L, or vice-versa.
3. Presence of clonal effacement, wherein lymphoid follicles are in various stages of replacement of normal non-clonal B-lymphocytes (e.g., with a K/L ratio of 2:1 K:L, or vice-versa) by malignant clonal B-lymphocytes (e.g., with a K/L ratio of 9:1 K:L, or vice-versa). Clonal effacement originates in the center of the follicle and progresses outwards to the periphery of the follicle.
4. Since the follicular centers consist of clonal population of malignant B-lymphocytes, the surrounding mantle-zone rim of normal non-clonal lymphocytes is more apparent when performing the RNA ISH for K/L. This is particularly true within 2-color assays for IgKC and IgLC, as the simultaneous detection facilitates an accurate and easy visual confirmation.
5. Presence of plasma cells (intensely stained cells) per lymphoid follicle below a threshold, e.g., less than 7, preferably less than 6, more preferably less than 5, and most preferably less than 4 plasma cells per follicle.
6. Malignant lymphoma cells will generally have larger and lighter hematoxylin stained nuclei. As described earlier, these cells will have a clonal IgKC/IgLC staining pattern. There may be presence of interspersed normal lymphocytes can be identified by their smaller size and darker hematoxylin stained nuclei. These cells will have a non-clonal IgKC/IgLC staining pattern. The exception is small cell lymphoma where malignant lymphoma cells have smaller and darker nuclei that are similar to normal lymphocytes. However these cells will have clonal IgKC/IgLC staining pattern.
   Additionally or alternatively, the further determination of Non-Hodgkin Lymphoma can be made based on one or more of the following morphological features seen by ISH:
1. IGKC+ Lymphoma cells show strong positive cytoplasmic ISH staining with IGKC probe and may show staining with IGLC probe predominantly in the nucleus due to the presence of IGL-Like targets (e.g., IGLL5). The presence of cytoplasmic IGKC ISH staining along with the nuclear IGLC staining may denote IGKC clonality in B-lymphocytes
2. IGLC+ Lymphoma cells show strong positive cytoplasmic ISH staining with IGLC probe and do not show staining with IGKC probe.

The detection of IgK+ and IgL+ cells can be performed using methods known in the art; a preferred method is RNA in situ hybridization (RNA ISH). Other methods known in the art for gene expression analysis, e.g., RT-PCR, RNA-sequencing, and oligo hybridization assays including RNA expression microarrays, hybridization based digital barcode quantification assays such as the nCounter® System (NanoString Technologies, Inc., Seattle, Wash.), and lysate based hybridization assays utilizing branched DNA signal amplification such as the QuantiGene® 2.0 Single Plex and Multiplex Assays (Affymetrix, Inc., Santa Clara, Calif.); however, these non-RNA ISH methods cannot visualize RNA in situ, which is important in identifying the cell of origin and the retention of cellular morphology and other aspects that are lost when cells are lysed. Thus in some embodiments of the methods described herein RNA ISH methods are used wherein the cells are individually identifiable (i.e., although the cells are permeabilized to allow for influx and outflux of detection reagents, the structure of individual cells is maintained such that each cell can be identified); in contrast, methods such as RT-PCR, expression arrays, and so on use bulk samples wherein the RNA is extracted from disrupted cells, and the cells are not identifiable (and thus the cell of origin cannot be identified).

Certain RNA ISH platforms leverage the ability to amplify the signal within the assay via a branched-chain technique of multiple polynucleotides hybridized to one another (e.g., bDNA) to form a branch structure (e.g., branched nucleic acid signal amplification). In addition to its high sensitivity, the platform also has minimal non-specific background signal compared to immunohistochemistry. While RNA ISH has been used in the research laboratory for many decades, tissue based RNA diagnostics have only recently been introduced in the diagnostic laboratory. However, these have been restricted to highly expressed transcripts such as immunoglobulin light chains as low abundance transcripts such as IgL otherwise cannot be detected by a conventional RNA ISH platform (Hong et al., Surgery 146:250-257, 2009; Magro et al., J Cutan Pathol 30:504-511, 2003). This robust RNA ISH platform with its ability to detect low transcript numbers has the potential to revolutionize RNA diagnostics in paraffin tissue and other tissue assay sample formats.

In some embodiments, the assay is a bDNA assay, optionally as described in U.S. Pat. Nos. 7,709,198; 7,803,541; 8,114,681 and 2006/0263769, which describe the general bDNA approach; see especially 14:39 through 15:19 of the '198 patent. In some embodiments, the methods include using a modified RNA in situ hybridization (ISH) technique using a branched-chain DNA assay to directly detect and evaluate the level of biomarker mRNA in the sample (see, e.g., Luo et al., U.S. Pat. No. 7,803,541B2, 2010; Canales et al., Nature Biotechnology 24(9):1115-1122 (2006); Ting et al., Aberrant Overexpression of Satellite Repeats in Pancreatic and Other Epithelial Cancers, Science 331(6017):593-6 (2011)). A kit for performing this assay is commercially-available from Affymetrix, Inc. (e.g., the ViewRNA™ Assays for tissue and cell samples).

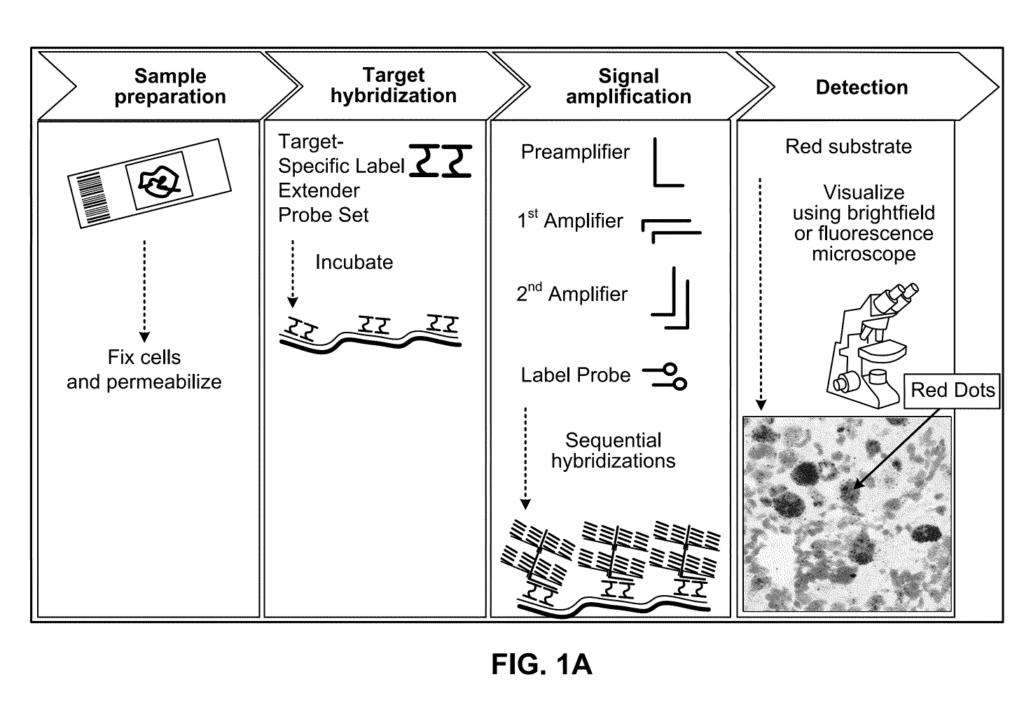

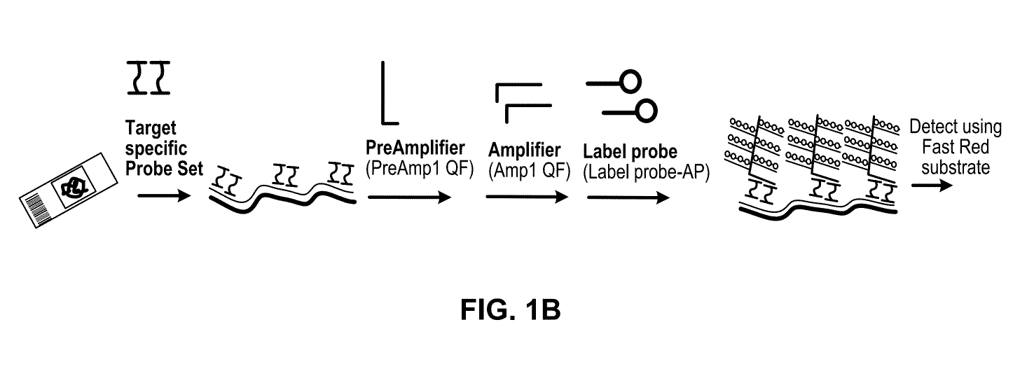

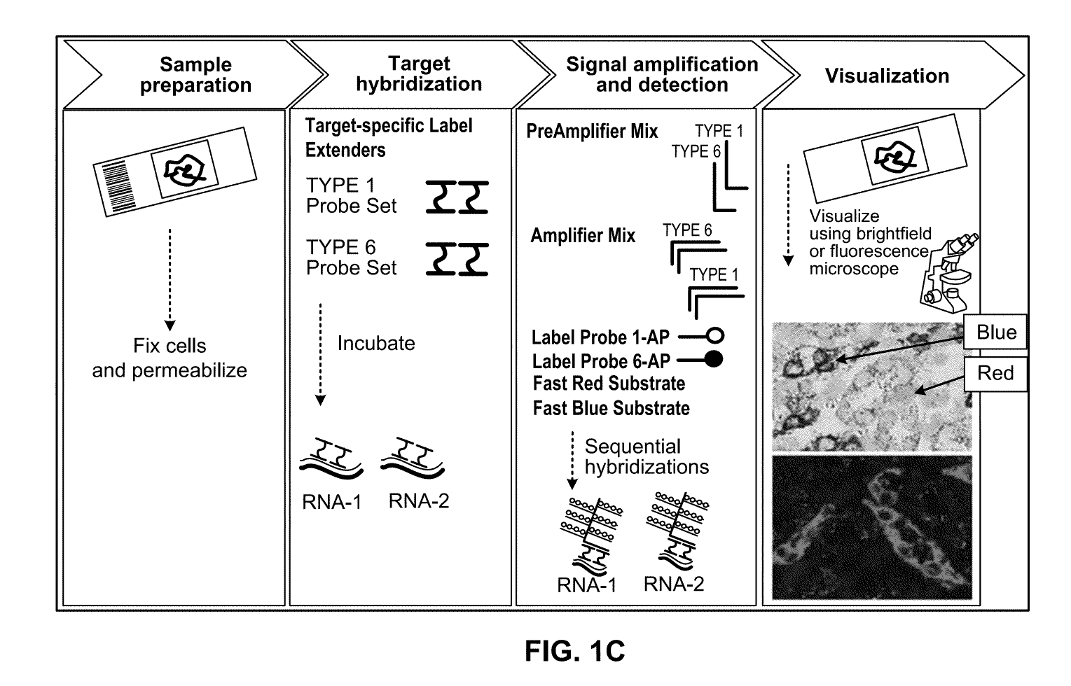

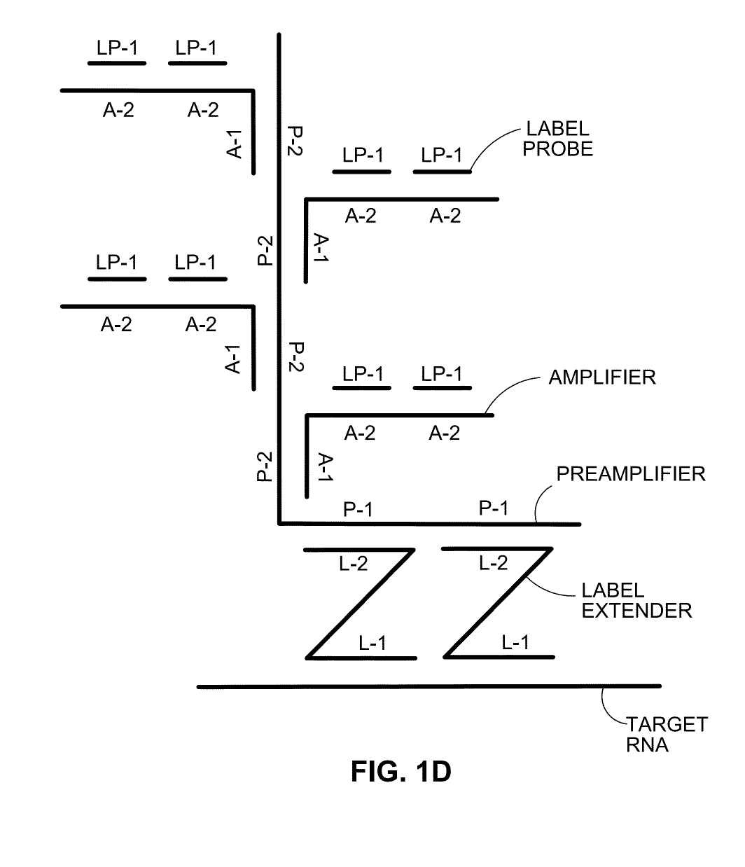

RNA ISH can be performed, e.g., using the ViewRNA™ technology (Affymetrix, Santa Clara, Calif.). ViewRNA™ ISH is based on the branched DNA technology wherein signal amplification is achieved via a series of sequential steps (e.g., as shown in FIGS. 1A-B in a single plex format and in FIG. 1C in a two plex format). Thus in some embodiments, the methods include performing an assay as described in US 2012/0052498 (which describes methods for detecting both a nucleic acid and a protein with bDNA signal amplification, comprising providing a sample comprising or suspected of comprising a target nucleic acid and a target protein; incubating at least two label extender probes each comprising a different L-1 sequence, an antibody specific for the target protein, and at least two label probe systems with the sample comprising or suspected of comprising the target nucleic acid and the target protein, wherein the antibody comprises a pre-amplifier probe, and wherein the at least two label probe systems each comprise a detectably different label; and detecting the detectably different labels in the sample); US 2012/0004132; US 2012/0003648 (which describes methods of amplifying a nucleic acid detection signal comprising hybridizing one or more label extender probes to a target nucleic acid; hybridizing a pre-amplifier to the one or more label extender probes; hybridizing one or more amplifiers to the pre-amplifier; hybridizing one or more label spoke probes to the one or more amplifiers; and hybridizing one or more label probes to the one or more label spoke probes); or US 2012/0172246 (which describes methods of detecting a target nucleic acid sequence, comprising providing a sample comprising or suspected of comprising a target nucleic acid sequence; incubating at least two label extender probes each comprising a different L-1 sequence, and a label probe system with the sample comprising or suspected of comprising the target nucleic acid sequence; and detecting whether the label probe system is associated with the sample). Each hybridized target specific polynucleotide probe acts in turn as a hybridization target for a pre-amplifier polynucleotide that in turn hybridizes with one or more amplifier polynucleotides. In some embodiments two or more target specific probes (label extenders) are hybridized to the target before the appropriate pre-amplifier polynucleotide is bound to the 2 label extenders, but in other embodiments a single label extender can also be used with a pre-amplifier. Thus, in some embodiments the methods include incubating one or more label extender probes with the sample. In some embodiments, the target specific probes (label extenders) are in a ZZ orientation, cruciform orientation, or other (e.g., mixed) orientation; see, e.g., FIGS. 10A and 10B of US 2012/0052498. Each amplifier molecule provides binding sites to multiple detectable label probe oligonucleotides, e.g., chromogen or fluorophore conjugated-polynucleotides, thereby creating a fully assembled signal amplification “tree” that has numerous binding sites for the label probe; the number of binding sites can vary depending on the tree structure and the labeling approach being used, e.g., from 16-64 binding sites up to 3000-4000 range. In some embodiments there are 300-5000 probe binding sites. The number of binding sites can be optimized to be large enough to provide a strong signal but small enough to avoid issues associated with overlarge structures, i.e., small enough to avoid steric effects and to fairly easily enter the fixed/permeabilized cells and be washed out of them if the target is not present, as larger trees will require larger components that may get stuck within pores of the cells (e.g., the pores created during permeabilization, the pores of the nucleus) despite subsequent washing steps and lead to noise generation. A non-limiting bDNA amplification scheme is shown in FIG. 1D.

In some embodiments, the label probe polynucleotides are conjugated to an enzyme capable of interacting with a suitable chromogen, e.g., alkaline phosphatase (AP) or horseradish peroxidase (HRP). Where an alkaline phosphatase (AP)-conjugated polynucleotide probe is used, following sequential addition of an appropriate substrate such as fast red or fast blue substrate, AP breaks down the substrate to form a precipitate that allows in-situ detection of the specific target RNA molecule. Alkaline phosphatase can be used with a number of substrates, e.g., fast red, fast blue, or 5-Bromo-4-chloro-3-indolyl-phosphate (BCIP). Thus in some embodiments, the methods include the use of alkaline phosphatase conjugated polynucleotide probes within a bDNA signal amplification approach, e.g., as described generally in U.S. Pat. No. 5,780,277 and U.S. Pat. No. 7,033,758. Other enzyme and chromogenic substrate pairs can also be used, e.g., horseradish peroxidase (HRP) and 3,3′-Diaminobenzidine (DAB). Many suitable enzymes and chromogen substrates are known in the art and can be used to provide a variety of colors for the detectable signals generated in the assay, and suitable selection of the enzyme(s) and substrates used can facilitate multiplexing of targets and labels within a single sample. For these embodiments, labeled probes can be detected using known imaging methods, e.g., bright-field microscopy with a CISH approach.

Other embodiments include the use of fluorophore-conjugates probes, e.g., Alexa Fluor dyes (Life Technologies Corporation, Carlsbad, Calif.) conjugated to label probes. In these embodiments, labeled probes can be detected using known imaging methods, e.g., fluorescence microscopy (e.g., FISH). Selection of appropriate fluorophores can also facilitate multiplexing of targets and labels based upon, e.g., the emission spectra of the selected fluorophores.

In some embodiments, the assay is similar to those described in US 2012/0100540; US 2013/0023433; US 2013/0171621; US 2012/0071343; or US 2012/0214152. All of the foregoing are incorporated herein by reference in their entirety.

In some embodiments, an RNA ISH assay is performed without the use of bDNA, and the IgK and IgL specific probes are directly or indirectly (e.g., via an antibody) labeled with one or more labels as discussed herein.

The assay can be conducted manually or on an automated instrument, such the Leica BOND family of instruments, or the Ventana DISCOVERY ULTRA or DISCOVERY XT instruments.

In some embodiments, the detection methods use an RNA probe set targeting the human IgK or IgL mRNA transcripts, e.g., as shown in FIGS. 1A-C. The presence of a ratio of IgK/IgL over a threshold, e.g., over 6:1, preferably over 7:1, more preferably over 8:1, or even more preferably over 9:1, indicates that the sample is likely to be from MM or NHL, while a ratio below that threshold indicates that it is not likely to be from MM or NHL; an exemplary decision tree is shown in FIG. 8A. As noted above, the levels of IgK and IgL can be determined in the same section, e.g., using a 2-plex assay with different labels, e.g., different chromogenic enzyme/substrate pairs (such as AP/fast red and HRP/DAB) (see FIG. 1C) or different fluorophores. Alternatively, the levels can be determined using a 1-plex assay in consecutive sections, e.g., using the same or different labels (see FIGS. 1A-B).

In some embodiments, the detection methods include detecting IgK and IgL in combination with one or more pan-housekeeping (pan-HKG) genes, e.g. GAPDH, ACTB, PPIB or UBC, to assess RNA integrity. Within some embodiments, a panel of two or more housekeeping genes is utilized to account for the expression of certain genes being affected by a particular disease or being innately different in the individual from which the sample was collected. To avoid unnecessarily requiring more distinct labels to be used within an assay, the measurement of the HKG panel of two or more HKGs may utilize a common label (e.g., to provide a common detectable signal such as a color in a CISH assay or a particular emission spectra in a FISH assay). Cells that do not have expression of pan-HKG lack essential RNA integrity and hence need to be excluded from the analysis; an exemplary decision tree is shown in FIG. 8B. This eliminates false negative cases, as may arise with, e.g., improperly stored or prepared samples.

For example, in an embodiment wherein IgK and IgL are detected in consecutive sections, the 1^{st} tissue section can be used to detect IgL and HKG, and the 2^{nd} tissue section to detect IgK and HKG. In an embodiment wherein IgK and IgL are determined in the same section, IgL, IgK and HKG are all determined in the same section, using three different labels. Both can be done in the same manner as the non-HKG tests, e.g., using chromogenic ISH (CISH) or fluorescence ISH (FISH). For CISH, one could use 3 different label probe systems, e.g., (1) alkaline phosphatase and fast red, (2) alkaline phosphatase and fast blue, and (3) horseradish peroxidase (HRP) and 3,3′-Diaminobenzidine (DAB). For FISH, an assay could employ 3 different fluorophores that have peak emissions with sufficient separation to allow distinct detection, such as peak emission values at, e.g., 519 nm, 665 nm, and 775 nm. Many suitable fluorophores are commercially available, e.g., Life Technologies offers Alexa Fluor dyes with peak emission values ranging from 442 nm to 814 nm, allowing straightforward fluorescent multiplexing.

Probes

Each probe set contains one or more, preferably multiple, polynucleotide probes (also referred to herein as label extenders for embodiments utilizing branched nucleic acid signal amplification). Preferably, each label extender probe consists of three parts with (1) part 1 designed to hybridize to the targeted gene, (2) part 2 being nucleotide spacer (e.g., 3-20 nucleotides) and (3) part 3 designed to hybridize to the unique tag within a bDNA preamplifier probe (see below and FIG. 1D).

| | | |
|---|---|---|
| Part1 | Part2 | Part3 |
| (binds to target region) | (spacer) | (binds to bDNA) |

nnnnnnnnnnnnnnnnnnnnnnnnnSSSSSSSSSSSSSSBBBBBBBBBBBBBBBBBBB

The Part1 sequence of a probe can span a wide variety of lengths, from 12 bases to the full length of the target sequence, and will vary depending on the intended target and overall assay design characteristics (e.g., the desired hybridization temperature). Within certain embodiments, the Part1 sequence is preferably from 16 bases to 32 bases in length. The probe set for IgK can range from 1 or 2 polynucleotides to e.g., 5, 10, 15, 20 polynucleotides or more, and the probe set for IgL can range from 1 or 2 polynucleotides to 5, 10, 15, 20, 25 polynucleotides or more, with the number of probes in each set depending on, e.g., the desired regions of each RNA target to be interrogated, the number of target regions desired in order to generate sufficient signal with the relevant detection approach of a particular assay, the contrast in total signal desired between IgL and IgK positive cells. In preferred embodiments, the Tm of each oligonucleotide is between 60° C. and 70° C.

The sequences of human IgK and IgL are known in the art. K is encoded by a locus on Chromosome 2p12 (GenBank Acc. No. NG_{—}000834.1), while L is encoded by a locus on Chromosome 22q11.2 (GenBank Acc. No. NG_{—}000002.1).

In preferred embodiments, the probes that bind to IgL mRNA bind to a unique (non-homologous) region of Homo sapiens Ig lambda chain, e.g., within the following sequence (preferably within the underlined region):

(SEQ ID NO: 1)
ggccagcttccctctcctcctcaccctcctcactcactgtgcagggtcct
gggcccagtctgtgctgactcagccaccctcagcgtctgggacccccggg
cagagggtcatcatctcttgttctggaagcagctccaacatcggaggtaa
tactgtaaactggtaccagcagctcccaggaagggcccccaaactcctca
tccatagtaataatcagcggccctcaggggtccctgaccgattctctggc
tccaagtctggcacctcagcctccctggccatcagtgggctccagtctga
ggatgaggctgattattactgtgcagcatgggatgacagcctgaatggtc
ggtatgtcttcggaactgggaccaaggtcaccgtcctaggtcagcccaag
gccaaccccactgtcactctgttcccgccctcctctgaggagctccaagc
caacaaggccacactagtgtgtctgatcagtgacttctacccgggagctg
tgacagtggcctggaaggcagatggcagccccgtcaaggcgggagtggag
accaccaaaccctccaaacagagcaacaacaagtacgcggccagcagcta
cctgagcctgacgcccgagcagtggaagtcccacagaagctacagctgcc
aggtcacgcatgaagggagcaccgtggagaagacagtggcccctacagaa
tgttcataggttcccaactctaaccccacccacgggagcctggagctgca
ggatcccaggggaggggtctctctccccatcccaagtcatccagcccttc
tccctgcactcatgaaacc.

In preferred embodiments, the probes that bind to IgK mRNA bind to a unique (non-homologous) region of the Homo sapiens Ig kappa chain, e.g., within the following sequence (preferably within the underlined region):

(SEQ ID NO: 2)
ggggagtcagtcccagtcaggacacagcatggacatgagggtccccgctc
agctcctggggctcctgctgctctggctcccaggtgccagatgtgacatc
cagttgacccagtctccatccttcctgtctgcagctgtgggagacagagt
cagcatcacttgccgggccagtcaggacatcagcaaatatttagcctggt
atcaacataaaattgggaaagcccctaaactcctgatctatggtgcatcc
actttgcaaagtggggtcccatcaagattcagtggcagtgggtctgggac
agaattcactctcacaatcaacagcctgcagcctgaggatctcgcaactt
attactgtcaacaacttaataattaccccctcactttcggcggggggacc
aaggtggagatcatacgaactgtggctgcaccatctgtcttcatcttccc
gccatctgatgagcagttgaaatctggaactgcctctgttgtgtgcctgc
tgaataacttctatcccagagaggccaaagtacagtggaaggtggataac
gccctccaatcgggtaactcccaggagagtgtcacagagcaggacagcaa
ggacagcacctacagcctcagcagcaccctgacgctgagcaaagcagact
acgagaaacacaaagtctacgcctgcgaagtcacccatcagggcctgagc
tcgcccgtcacaaagagcttcaacaggggagagtgttagagggagaagtg
cccccacctgctcctcagttccagcctgaccccctcccatcctttggcct
ctgaccctttttccacaggggacctacccctattgcggtcctccagctca
tctttcacctcacccccctcctcctccttggctttaattatgctaatgtt
ggaggagaatgaataaataaagtgaatctttgcacctgcaaaaaaaaaaa
aaaaaaaaaaaaaaaaaaa.

Exemplary target-specific regions (e.g., Part 1 as described above) for the probe sets for IgKC and IgKL are shown in Table B.

TABLE B

| IGKC | | SEQ ID |
|---|---|---|
| Probe | Sequence | NO: |
| 1 | tgaagacagatggtgcagcca | 3 |
| 2 | ctcatcagatggcgggaaga | 4 |
| 3 | gaggcagttccagatttcaactg | 5 |
| 4 | agttattcagcaggcacacaaca | 6 |
| 5 | actttggcctctctgggataga | 7 |
| 6 | gcgttatccaccttccactgt | 8 |
| 7 | gggagttacccgattggagg | 9 |
| 8 | gtcctgctctgtgacactctcct | 10 |
| 9 | gcgtcagggtgctgctgag | 11 |
| 10 | tttctcgtagtctgctttgctca | 12 |
| 11 | tcgcaggcgtagactttgtg | 13 |
| 12 | caggccctgatgggtgact | 14 |
| IGLC | | |
| Probe | Sequence | |
| 1 | gcgggaacagagtgacagtgg | 15 |
| 2 | cttggagctcctcagaggagg | 16 |
| 3 | cacactagtgtggccttgttgg | 17 |
| 4 | ccgggtagaagtcactgatcaga | 18 |
| 5 | ccaggccactgtcacagctc | 19 |
| 6 | ggggctgccatctgcctt | 20 |
| 7 | tctccactcccgccttgac | 21 |
| 8 | ctgtttggagggtttggtgg | 22 |
| 9 | cctggcagctgtagcttctgtg | 23 |
| 10 | gtgctcccttcatgcgtga | 24 |
| 11 | gggccactgtcttctccacg | 25 |
| 12 | ttgggaacctatgaacattctgtag | 26 |
| 13 | cccgtgggtggggttagag | 27 |
| 14 | gatcctgcagctccaggct | 28 |

In some embodiments, the one or more polypeptide probes that bind specifically to IgK mRNA in situ are selected from Table B. Additionally or alternatively, the one or the one or more polypeptide probes that bind specifically to IgL mRNA in situ are selected from Table B.

One of skill in the art would readily be able to identify sequences for additional species bioinformatically, and would appreciate that the sequence of IgK and IgL mRNA used should match the species of the subject from which the sample is obtained. The subject is preferably a mammal and can be, e.g., a human or veterinary subject (e.g., cat, dog, horse, cow, or sheep).

Treatment

In some embodiments of the methods described herein, once a subject has been identified as having MM, HL, or NHL, a treatment as known in the art can be administered.

Treatment for MM typically includes Chemotherapy (e.g., with Melphalan; Vincristine (Oncovin®); Cyclophosphamide (Cytoxan®); Etoposide (VP-16); Doxorubicin (Adriamycin®); Liposomal doxorubicin (Doxil®); or Bendamustine (Treanda®); Bisphosphonates (e.g., pamidronate (Aredia®) or zoledronic acid (Zometa®)) or other drugs (e.g., corticosteroids such as dexamethasone and prednisone; immunomodulating agents such as thalidomide or lenalidomide or pomalidomide; Proteasome inhibitors such as Bortezomib (Velcade®) or Carfilzomib (Kyprolis™)), or combinations thereof (e.g., Melphalan and prednisone (MP), with or without thalidomide or bortezomib; Vincristine, doxorubicin (Adriamycin), and dexamethasone (called VAD); Thalidomide (or lenalidomide) and dexamethasone; Bortezomib and dexamethasone, with or without doxorubicin or thalidomide; Liposomal doxorubicin, vincristine, dexamethasone; Carfilzomib;

Dexamethasone, cyclophosphamide, etoposide, and cisplatin (called DCEP); or Dexamethasone, thalidomide, cisplatin, doxorubicin, cyclophosphamide, and etoposide (called DT-PACE), with or without bortezomib); Radiation (e.g., external beam radiation therapy); Surgery; Biologic therapy (e.g., with Interferon, Erythropoietin (Procrit®) or darbepoietin (Aranesp®)); Stem cell transplant (e.g., autologous or allogeneic peripheral blood stem cell transplant, bone marrow transplant, or cord blood transplant); or Plasmapheresis.

The main types of treatment for non-Hodgkin lymphoma include chemotherapy (e.g., with Cyclophosphamide (Cytoxan®); Vincristine (Oncovin®); Doxorubicin (Adriamycin®); Prednisone; Fludarabine (Fludara®); Cytarabine (ara-C); Chlorambucil; Mitoxantrone; Methotrexate; Etoposide (VP-16); Dexamethasone (Decadron®); Cisplatin; Carboplatin; Ifosfamide (Ifex®); Bleomycin; Bendamustine (Treanda®); Gemcitabine (Gemzar®); or Pralatrexate (Folotyn®)), or other drugs (e.g. Bortezomib (Velcade®), Romidepsin (Istodax®), or Ibrutinib (Imbruvica™)), or combinations thereof, e.g., cyclophosphamide, doxorubicin, vincristine and prednisone); radiation (e.g., external beam radiation); immunotherapy (e.g., with monoclonal antibodies such as Rituximab (Rituxan®), Alemtuzumab (Campath®), Ofatumumab (Arzerra®), or Brentuximab vedotin (Adcetris®); interferon; or immunomodulating agents such as thalidomide or lenalidomide); and High-dose chemotherapy and stem cell transplant (e.g., autologous or allogeneic peripheral blood stem cell transplant, bone marrow transplant, or cord blood transplant). Surgery is rarely used.

Treatment for HL includes chemotherapy, radiation, immunotherapy, and stem-cell transplant, or combinations thereof, e.g., as described above for NHL. In some embodiments, treatment for HL can include one or more of the following regimens: MOPP (mechlorethamine, vincristine, procarbazine, prednisone); ABVD (Adriamycin [doxorubicin], bleomycin, vinblastine, dacarbazine); Stanford V (doxorubicin, vinblastine, mustard, bleomycin, vincristine, etoposide, prednisone); or BEACOPP (bleomycin, etoposide, doxorubicin, cyclophosphamide, vincristine, procarbazine, prednisone).

MALT lymphoma that is confined to the stomach may be treated with antibiotics to eradicate H. pylori (see, e.g., Bayerdorffer et al., (1995) Lancet 345 (8965): 1591-4.

For additional information about appropriate treatments, see, e.g., the NCCN cancer treatment guidelines; ASCO treatment guidelines; ESMO treatment guidelines; Oxford Textbook of Oncology, Second Edition; Textbook of Medical Oncology, Informa Healthcare; Comprehensive Textbook of Oncology.

Kits

There are provided herein kits comprising reagents for performing any of the methods described herein. In some embodiments, a kit comprises one or more polynucleotide probes that are capable of binding specifically to IgK mRNA in situ and one or more polynucleotide probes that are capable of binding specifically to IgL mRNA in situ.

In some embodiments, a kit comprises one or more label extender probes that are capable of binding to one or more target regions in the IgK mRNA and one or more label extender probes that are capable of binding to one or more target regions in the IgL mRNA.

In some embodiments the one or more polynucleotide probes that are capable of binding specifically to IgK mRNA in situ comprise one or more label extender probes that are capable of binding to one or more target regions in the IgK mRNA, one or more pre-amplifier probes that are capable of hybridizing to the one or more label extender probes, one or more amplifier probes that are capable of hybridizing to the one or more pre-amplifier probes, and one or more label probes that are capable of hybridizing to the one or more amplifier probes.

Additionally or alternatively, in some embodiments the one or more polynucleotide probes that are capable of binding specifically to IgL mRNA in situ comprise one or more label extender probes that are capable of binding to one or more target regions in the IgL mRNA, one or more pre-amplifier probes that are capable of hybridizing to the one or more label extender probes, one or more amplifier probes that are capable of hybridizing to the one or more pre-amplifier probes, and one or more label probes that are capable of hybridizing to the one or more amplifier probes.

In some embodiments the kit further comprises one or more polynucleotide probes that bind specifically to mRNA encoding a housekeeping gene (HKG) in situ. In some embodiments, the kit comprises one or more label extender probes that are capable of binding to one or more target regions in the HKG mRNA

In some embodiments, the one or more polynucleotide probes that are capable of binding specifically to mRNA encoding a HKG in situ comprise one or more label extender probes that are capable of binding to one or more target regions in the HKG mRNA, one or more pre-amplifier probes that are capable of hybridizing to the one or more label extender probes, one or more amplifier probes that are capable of hybridizing to the one or more pre-amplifier probes, and one or more label probes that are capable of hybridizing to the one or more amplifier probes.

### Examples

The invention is further described in the following examples, which do not limit the scope of the invention described in the claims.

Statistical Analysis

Statistics were calculated using SPSS version 21.0 (SPSS, Chicago, Ill., USA). Differences between groups were evaluated using the Student t-test for quantitative variables. A P-value <0.05 was considered significant.

### Example 1

### Detection of Clonal Populations of IgK/IgL Expressing Plasma Cells and B-Lymphocytes Using RNA ISH

Branched chain DNA (bDNA) in situ hybridization (ISH) was performed using the ViewRNA™ technology (Affymetrix, Santa Clara, Calif.). ViewRNA™ in situ hybridization is based on the branched DNA technology wherein signal amplification is achieved via a series of sequential steps. Each pair of bound target probe set oligonucleotides acts a template to hybridize a pre-amplifier molecule that in turn binds multiple amplifier molecules. Each amplifier molecule provides binding sites to multiple alkaline phosphatase (AP)-conjugated-oligonucleotides thereby creating a fully assembled signal amplification “tree” that has approximately 400 binding sites for the AP-labeled probe. Following sequential addition of the substrate (e.g., fast red or fast blue), AP breaks down the substrate to form a precipitate (red dots in the case of fast red, blue dots for fast blue) that allows in-situ detection of the specific target RNA molecule.

In situ hybridization probes (Affymetrix, Santa Clara, Calif.) were designed against IgK and IgL transcript; IgK is encoded by a locus on Chromosome 2p12 (GenBank Acc. No. NG_{—}000834.1), while IgL is encoded by a locus on Chromosome 22q11.2 (GenBank Acc. No. NG_{—}000002.1). See Table B for exemplary target-specific sequences.

These probe sets were used in conjunction with the ViewRNA™ Tissue Assay Kit (2-plex) and in situ hybridization was performed according to the manufacturer's instructions. Briefly, dissected tissues were fixed for <24 hours in 10% Neutral Buffer Formalin at room temperature, followed by the standard formaldehyde-fixed, paraffin-embedded (FFPE) preparation. The FFPE tissues were sectioned at 5+/−1 micron and mounted on Surgipath X-tra glass slide (Leica BioSystems, Buffalo Grove, Ill.), baked for 1 hour at 60° C. to ensure tissue attachment to the glass slides, and then subjected to xylene deparaffinization and ethanol dehydration. To unmask the RNA targets, dewaxed sections were incubated in 500 ml pretreatment buffer (Affymetrix/Santa Clara, Calif.) at 90-95° C. for 10 minutes and digested with 1:100 dilution protease at 40° C. (Affymetrix, Santa Clara, Calif.) for 10 minutes, followed by fixation with 10% formaldehyde at room temperature for 5 minutes. Unmasked tissue sections were subsequently hybridized with 1:40 dilution IgL or IgK probe sets for 2 hours at 40° C., followed by series of post-hybridization washes. Signal amplification was achieved via a series of sequential hybridizations and washes as described in the user's manual. Slides were post-fixed with 4% formaldehyde, counterstained with Gill's hematoxylin, mounted using Advantage Mounting Media (Innovex, Richmond, Calif.), and visualized using a standard bright-field microscope.

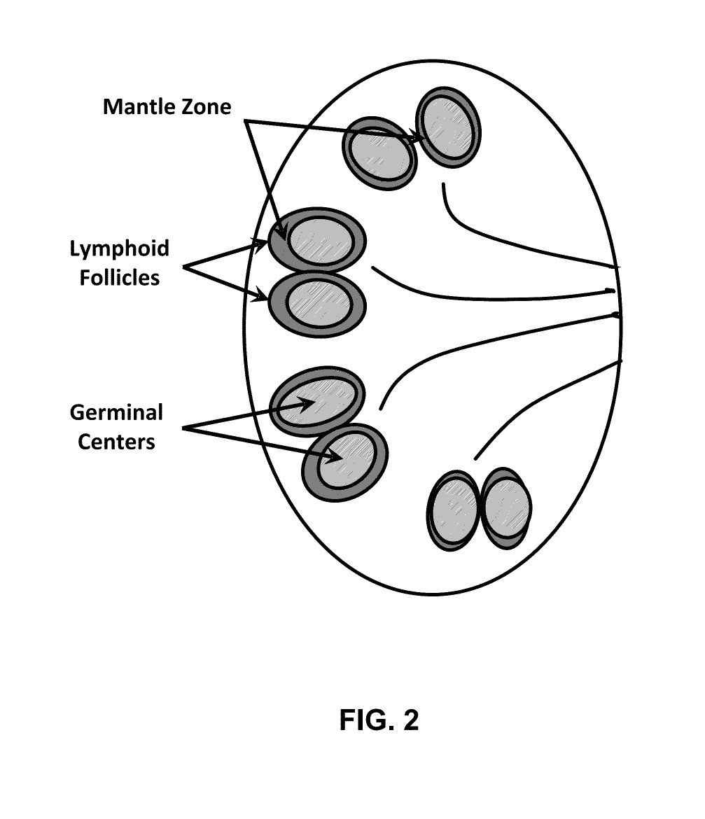

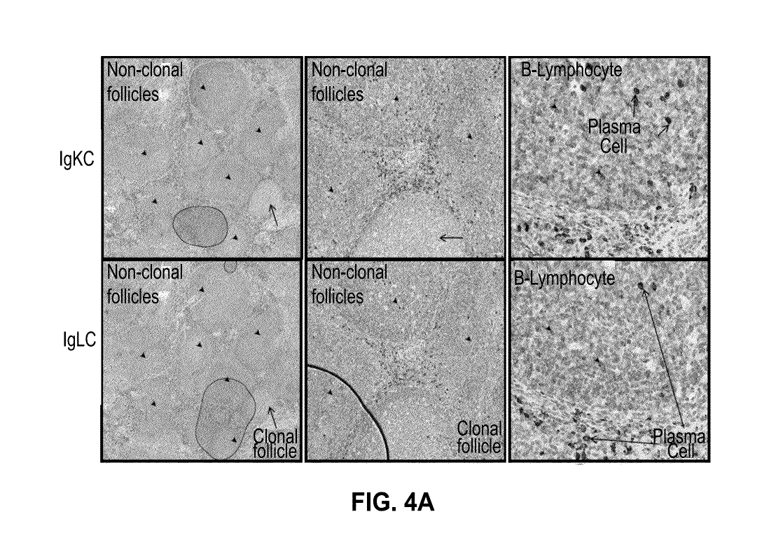

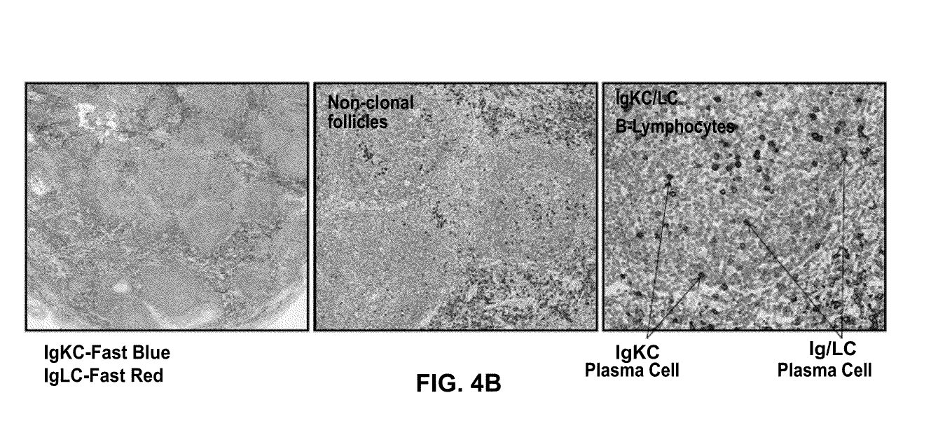

Results were obtained from reactive and normal lymph node tissue (IGKC-IGLC non-clonal) using 1-plex ViewRNA™ ISH (FIG. 4A) and 2-plex ViewRNA™ ISH (FIG. 4B). The left panel of FIG. 4A is a low power view of reactive lymph node showing multiple lymphoid follicles (arrowheads) positively stained for IGKC and IGLC RNA ISH stain (red in original). Interestingly, there appeared to be one follicle that had presence of IGLC staining but absence of IGKC ISH stain (arrow). Occasionally clonal follicles may be observed in reactive lymph nodes. The center panel of FIG. 4A is RNA ISH showing presence of both IGKC and IGLC RNA ISH staining within follicles (light red in original) and interfollicular tissue showing presence of dark red staining cells. The right panel of FIG. 4A is a high power view showing IGKC and IGLC RNA ISH staining in B-lymphocytes (arrowheads) within the lymphoid follicles. The IGKC and IGLC-bearing B-lymphocytes were distributed in roughly equal proportions. There were multiple dark-red staining plasma cells present within lymphoid follicles and in the interfollicular tissue (arrows).

The left panel of FIG. 4B is a low power view of reactive lymph node showing 2-plex RNA ISH stain for IGKC (red in original) and IGLC (blue in original). RNA ISH showed an approximately equal proportion of IGKC (red in original) and IGLC (blue in original) staining in the lymphoid follicles (arrowhead). The interfollicular tissue showed the presence of dark red staining, non-clonal IGKC (red in original) and IGLC (blue in original) bearing plasma cells. The right panel of FIG. 4B is a high power view showing IGKC (red in original) and IGLC (blue in original) RNA ISH staining in B-lymphocytes (arrowheads) within the lymphoid follicles. Again, there was approximately equal proportion of IGKC and IGLC bearing cells. There was no predominance of one type of light-chain bearing cell over the other which signifies non-clonality. The figure shows the presence dark-red/dark-blue staining plasma cells (arrows) within the lymphoid follicles and in the interfollicular areas. Generally there are greater numbers of plasma cells within reactive lymphoid follicles. The follicle shown in the image seems to lack the germinal center, where plasma cells are expected to be seen.

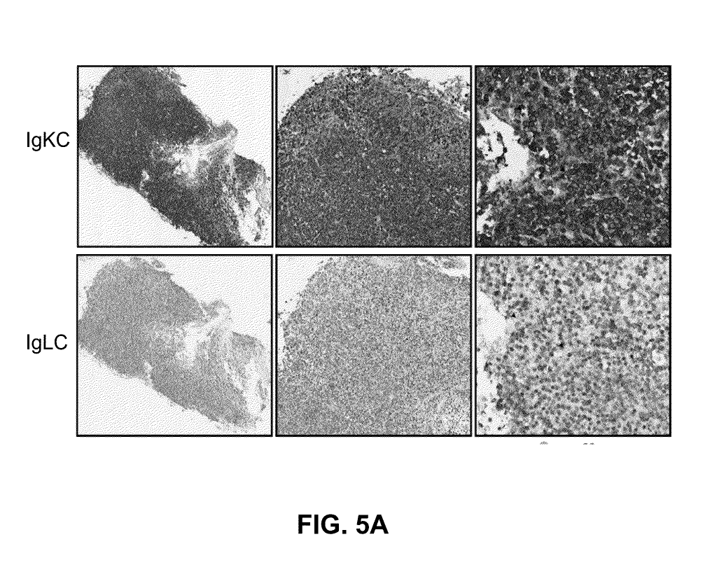

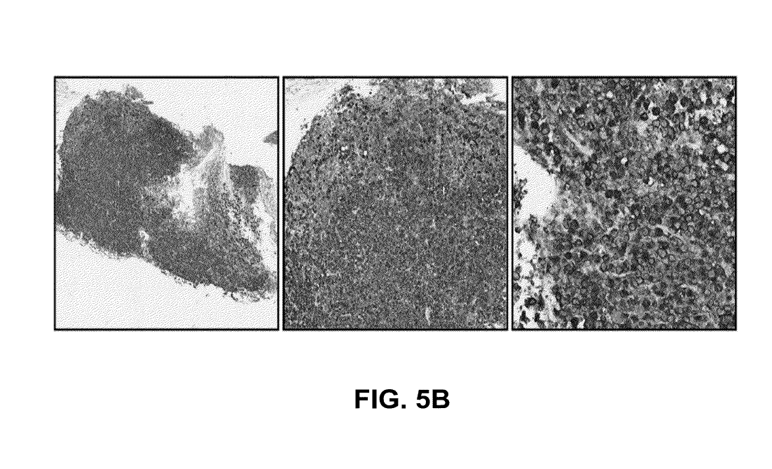

The left panel of FIG. 5A shows 1-plex IGKC RNA ISH stain (red in original) in a case of Multiple Myeloma showing presence, exclusively, of intense red staining plasma cells. The predominance of plasma cells expressing one type of Ig light chain (either K or L) signifies malignant plasma cell neoplasm (multiple myeloma). The right panel of FIG. 5A shows 1-plex IGLC RNA ISH stain (red in original) for the same tissue showing lack of intense red staining of plasma cells. There appeared to be a low level of IGLC staining in the tissue, presumably due to the homology of IGLC RNA ISH probe to IGLC-like 5 mRNA transcripts which can be expressed by clonal IGKC-plasma cells.

The left panel of FIG. 5B is a low power view of multiple myeloma using a 2-plex ViewRNA™ ISH with IGKC (blue in original) and IGLC (red in original). The tissue showed the presence, exclusively, of intense blue staining plasma cells. The right panel of FIG. 5B is a high power view of the same tissue showing predominance of intense-blue staining IGKC plasma cells (blue in original). One intense-red staining IGLC plasma cell was seen (arrow). The predominance of plasma cells expressing one type of Ig light chain (either K or L) signifies malignant plasma cell neoplasm (multiple myeloma). The low level of IGLC staining due to the homology of IGLC RNA ISH probe to IGLC-like 5 mRNA transcripts was not apparent in a 2-plex ISH mode due to the intense blue staining of the IGKC transcripts.

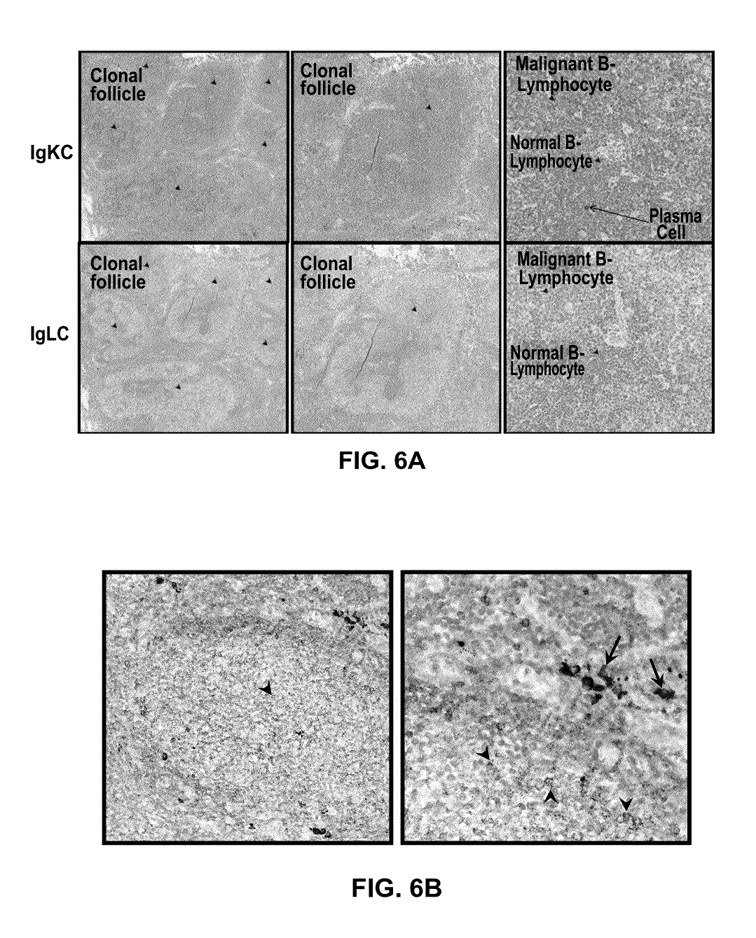

The left panel of FIG. 6A is a low power view of follicular lymphoma showing multiple lymphoid follicles (arrowheads) positively stained for IGKC (red in original) and lack of IGLC RNA ISH stain. The predominance of cells expressing one type of Ig light chain (either K or L) signified clonality. The center panel of FIG. 6A is RNA ISH showing light chain expression in follicles restricted to IGKC (arrowhead). There appeared to be no IGKC staining in the malignant follicles. The interfollicular tissue showed the presence of dark red staining cells, non-clonal IGKC and IGLC bearing plasma cells. The right panel of FIG. 6A is a high power view showing IGKC RNA ISH staining in B-lymphocytes (arrowheads) within the lymphoid follicles. There was no apparent IGLC staining in the malignant B-lymphocytes (arrowheads). The follicular center may show equal proportion of IGKC and IGLC-bearing B-cells representing non-clonal B-lymphocytes. There were very few/lack of dark-red staining plasma cells present within the malignant follicles (arrow).

The left panel of FIG. 6B is a low power view of follicular lymphoma showing 2-plex RNA ISH stain for IGKC (red in original) and IGLC (blue in original). RNA ISH showed light chain expression in follicles restricted to IGKC (red in original) and lack of IGLC (blue in original) staining in the malignant follicle (arrowhead). The predominance of cells expressing one type of Ig light chain (either K or L) signified clonality. The peripheral mantle zone showed equal proportion of IGKC (red in original) and IGLC (blue in original) B-cells representing non-clonal B-lymphocytes. The interfollicular tissue showed the presence of dark red staining, non-clonal IGKC (red in original) and IGLC (blue in original) bearing plasma cells. The right panel of FIG. 6B is a high power view showing IGKC (red in original) RNA ISH staining in B-lymphocytes (arrowheads) within the lymphoid follicles. There was no apparent IGLC (blue in original) staining in the malignant B-lymphocytes. The peripheral mantle zone showed an equal proportion of IGKC (red in original) and IGLC (blue in original) B-cells representing non-clonal B-lymphocytes. In addition, there were dark-red/dark-blue staining plasma cells in the interfollicular areas and very few/lack thereof within the malignant follicles (arrow).

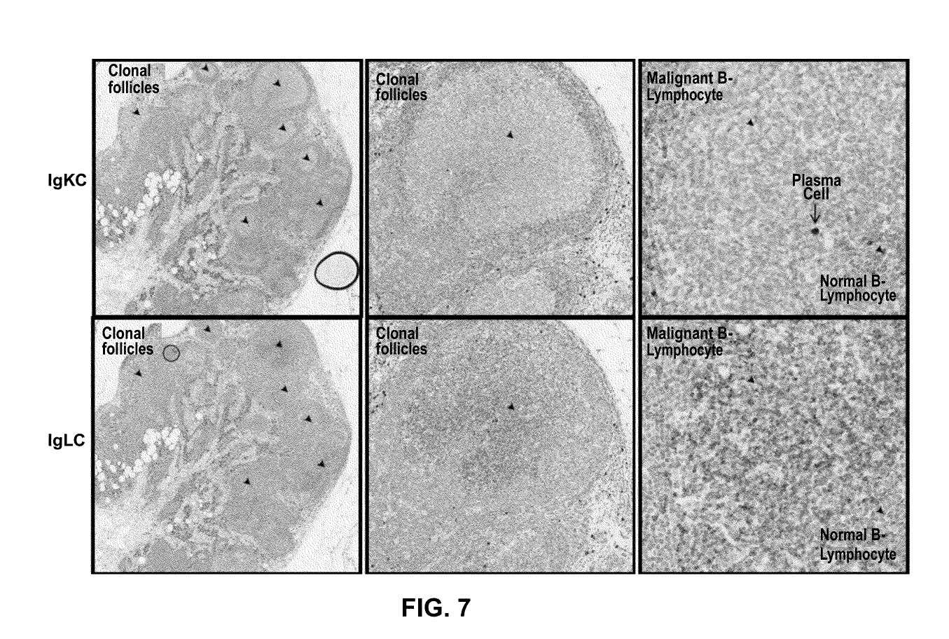

The left panel of FIG. 7 is a low power view of follicular lymphoma showing multiple lymphoid follicles (arrowheads) positively stained for IGLC (red in original) and lack of IGKC RNA ISH stain. The predominance of cells expressing one type of Ig light chain (either K or L) signified clonality. The center panel of FIG. 7 is RNA ISH showing light chain expression in follicles restricted to IGLC (arrowhead). There was a lack of IGKC staining in the malignant follicles. The peripheral mantle zone showed roughly equal proportions of IGKC and IGLC-bearing B-cells representing non-clonal B-lymphocytes (more apparent in IGKC). The interfollicular tissue showed the presence of dark red staining, non-clonal IGKC and IGLC bearing plasma cells. The right panel of FIG. 7 is a high power view showing IGLC RNA ISH staining in B-lymphocytes (arrowheads) within the lymphoid follicles. There was no apparent IGKC staining in the malignant B-lymphocytes (arrowheads), and very few/lack of dark-red staining plasma cells present within the malignant follicles (arrow).

### Example 2

### Detection of Clonal Populations of IgK/IgL Expressing Plasma Cells Using RNA ISH

In the present example, RNA-ISH stains for IgK and IgL were validated in a cohort of 23 clinically and pathologically confirmed patients with lymphoma and 14 reactive lymphoid controls. The lymphoma samples were enriched for Mucosa-Associated Lymphoid Tissue (MALT) lymphoma as it is frequently extranodal with admixed reactive lymphoid populations and may be less likely to have concurrent flow cytometry. bDNA ISH was performed as described in Example 1.

ISH results were interpreted separately by two observers blinded to ancillary testing results, and a third observer adjudicated in cases not fully concordant.

The results showed that a monoclonal or predominant light chain expression pattern with ISH was seen in 96% (22/23) of the lymphoma cases, corroborating ancillary studies. One MALT lymphoma (Case 4) appeared polytypic by ISH, but flow cytometry showed a small monoclonal B-cell population (Table 1). The 14 control cases were polytypic by ISH.

TABLE 1
ISH Compared To Ancillary Studies

| | | ISH | ISH | | |
|---|---|---|---|---|---|
| Case | Diagnosis | new | traditional | IHC | Flow |
| 1 | MALT | Kp | | Kp | MK |
| 2 | MALT | Kp | P | | MK |
| 3 | MALT | ML | P | | ML |
| 4 | MALT | P | | P | MK |
| 5 | MALT | MK | | MK | MK |
| 6 | MALT | ML | | | ML |
| 7 | MALT | Kp | Kp | | MK |
| 8 | MALT | ML | | ML | ML |
| 9 | MALT | MK | MK | | MK |
| 10 | MALT | Kp | Kp | | |
| 11 | MALT | MK | | MK | MK |
| 12 | MALT | MK | | | MK |
| 13 | MALT | Kp | | | MK |
| 14 | MALT | ML | | | ML |
| 15 | MALT | MK | | MK | MK |
| 16 | MALT | ML | ML | | |
| 17 | MALT | MK | MK | | MK |
| 18 | MALT | ML | Lp | | |
| 19 | FL | MK | P | | MK |
| 20 | FL | MK | | Ind: ?K | Ind: ?K |
| 21 | FL | ML | | | ML |
| 22 | EBV+ B-cell lymphoma | ML | ML | ML | ML |
| 23 | Clonal B-cells in | MK | | P | MK |
| | sialadenitis | | | | |

Abbreviations: ISH, in situ hybridization; IHC, immunohistochemistry; Kp, kappa predominant; MK, monoclonal kappa; P, polyclonal; ML, monoclonal lambda, Lp, lambda predominant, Ind: ?K, indeterminant/possibly kappa.

These results showed that branched chain ISH techniques can be used to determine clonality of B-cell populations. This method is particularly useful in cases with limited tissue and when flow cytometry is not available.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of diagnosing multiple myeloma (MM), Hodgkin Lymphoma (HL), or non-Hodgkin lymphoma (NHL) in a subject, the method comprising:
contacting a sample comprising cells from the subject with one or more polynucleotide probes that bind specifically to IgL mRNA in situ, and one or more polynucleotide probes that bind specifically to IgK mRNA in situ;
detecting binding of the probes to IgL mRNA and IgK mRNA in cells in the sample, to determine numbers of IgL-expressing cells and IgK-expressing cells;
calculating a ratio of IgL-expressing cells to IgK-expressing cells;
identifying the IgK-expressing cells and IgL-expressing cells as plasma cells or B-lymphocytes, and:
identifying a sample in which the ratio of IgL-expressing plasma cells to IgK-expressing plasma cells, or ratio of IgK-expressing plasma cells to IgL-expressing plasma cells, is above a threshold as being associated with MM; or
identifying a sample in which the ratio of IgL-expressing B-lymphocytes to IgK-expressing B-lymphocytes, or ratio of IgK-expressing B-lymphocytes to IgL-expressing B-lymphocytes, is above a threshold as being associated with NHL;
or
identifying a sample in which the ratio of IgL-expressing cells to IgK-expressing cells, or ratio of IgK-expressing cells to IgL-expressing cells, is below a threshold as not being associated with MM or NHL; or
identifying a sample with a mixture of light chain expressing and non-light chain expressing cells; with IgK and IgL expression present in the cytoplasm; and the presence of characteristic Reed Sternberg (RS) cells as being associated with HL.

2. A method of selecting a treatment for a subject suspected of having multiple myeloma (MM), Hodgkin Lymphoma (HL), or non-Hodgkin lymphoma (NHL), the method comprising:
contacting a sample comprising cells from the subject with one or more polynucleotide probes that bind specifically to IgL mRNA in situ, and one or more polynucleotide probes that bind specifically to IgK mRNA in situ;
detecting binding of the probes to IgL mRNA and IgK mRNA in cells in the sample, to determine numbers of IgL-expressing cells and IgK-expressing cells;
calculating a ratio of IgL-expressing cells to IgK-expressing cells;
identifying the IgK-expressing cells and IgL-expressing cells as plasma cells or B-lymphocytes, and:
identifying a sample in which the ratio of IgL-expressing plasma cells to IgK-expressing plasma cells, or ratio of IgK-expressing plasma cells to IgL-expressing plasma cells, is above a threshold as being associated with MM, and selecting a treatment for MM for the subject; or
identifying a sample in which the ratio of IgL-expressing B-lymphocytes to IgK-expressing B-lymphocytes, or ratio of IgK-expressing B-lymphocytes to IgL-expressing B-lymphocytes, is above a threshold as being associated with NHL, and selecting a treatment for NHL for the subject; or
identifying a sample with a mixture of light chain expressing and non-light chain expressing cells; with IgK and IgL expression present in the cytoplasm; and the presence of characteristic Reed Sternberg (RS) cells as being associated with HL, and selecting a treatment for HL for the subject;
or
identifying a sample in which the ratio of IgL-expressing cells to IgK-expressing cells, or ratio of IgK-expressing cells to IgL-expressing cells, is below a threshold as not being associated with MM or NHL, and optionally not treating the subject.

3. A method of treating a subject suspected of having multiple myeloma (MM), Hodgkin Lymphoma (HL), or non-Hodgkin lymphoma (NHL), the method comprising:
contacting a sample comprising cells from the subject with one or more polynucleotide probes that bind specifically to IgL mRNA in situ, and one or more polynucleotide probes that bind specifically to IgK mRNA in situ;
detecting binding of the probes to IgL mRNA and IgK mRNA in cells in the sample, to determine numbers of IgL-expressing cells and IgK-expressing cells;
calculating a ratio of IgL-expressing cells to IgK-expressing cells;
identifying the IgK-expressing cells and IgL-expressing cells as plasma cells or B-lymphocytes, and:
identifying a sample in which the ratio of IgL-expressing plasma cells to IgK-expressing plasma cells, or ratio of IgK-expressing plasma cells to IgL-expressing plasma cells, is above a threshold as being associated with MM, and administering a treatment for MM to the subject;
identifying a sample in which the ratio of IgL-expressing B-lymphocytes to IgK-expressing B-lymphocytes, or ratio of IgK-expressing B-lymphocytes to IgL-expressing B-lymphocytes, is above a threshold as being associated with NHL, and administering a treatment for NHL to the subject;
identifying a sample with a mixture of light chain expressing and non-light chain expressing cells; with IgK and IgL expression present in the cytoplasm; and the presence of characteristic Reed Sternberg (RS) cells as being associated with HL, and
administering a treatment for HL to the subject
or
identifying a sample in which the ratio of IgL-expressing cells to IgK-expressing cells, or ratio of IgK-expressing cells to IgL-expressing cells, is below a threshold as not being associated with MM or NHL, and optionally not treating the subject.

4. A method of making a differential diagnosis between multiple myeloma (MM), Hodgkin Lymphoma (HL), and non-Hodgkin lymphoma (NHL) in a subject, the method comprising:
contacting a sample comprising cells from the subject with one or more polynucleotide probes that bind specifically to IgL mRNA in situ, and one or more polynucleotide probes that bind specifically to IgK mRNA in situ;
detecting binding of the probes to IgL mRNA and IgK mRNA in cells in the sample, to determine numbers of IgL-expressing cells and IgK-expressing cells;
calculating a ratio of IgL-expressing cells to IgK-expressing cells;
identifying the IgK-expressing cells and IgL-expressing cells as plasma cells or B-lymphocytes, and:
diagnosing a subject in which the ratio of IgL-expressing plasma cells to IgK-expressing plasma cells, or ratio of IgK-expressing plasma cells to IgL-expressing plasma cells, is above a threshold as having MM;
diagnosing a subject in which a mixture of light chain expressing and non-light chain expressing cells with IgK and IgL expression present in the cytoplasm is present, and characteristic Reed Sternberg (RS) cells are present, as having HL; or
diagnosing a subject in which the ratio of IgL-expressing B-lymphocytes to IgK-expressing B-lymphocytes, or ratio of IgK-expressing B-lymphocytes to IgL-expressing B-lymphocytes, is above a threshold as having NHL.

5. The method of claim 1, wherein the sample is a biopsy sample obtained from the subject, and preferably wherein the sample comprises a plurality of individually identifiable cells.

6. The method of claim 5, wherein the sample has been fixed, embedded in a matrix, and sliced into sections.

7. The method of claim 6, wherein:
(i) the one or more polynucleotide probes that bind specifically to IgL mRNA in situ, and the one or more polynucleotide probes that bind specifically to IgK mRNA in situ, are both applied to a single section from the sample, or
(ii) the one or more polynucleotide probes that bind specifically to IgL mRNA in situ, and the one or more polynucleotide probes that bind specifically to IgK mRNA in situ, are applied to consecutive sections from the sample.

8. The method of claim 7, wherein:
the one or more polynucleotide probes that bind specifically to IgL mRNA in situ, and the one or more polynucleotide probes that bind specifically to IgK mRNA in situ, are both applied to a single section from the sample, and
binding of the one or more polynucleotide probes to IgL is detected using a first detectable signal, and binding of the one or more polynucleotide probes to IgK is detected using a second detectable signal.

9. The method of claim 1, wherein binding of the probes to IgL mRNA and IgK mRNA is detected using imaging, and wherein at least three high power fields (HPF) in the mass are analyzed to determine the number of IgL-positive and IgK-positive cells.

10. The method of claim 1, comprising one or both of:
detecting binding of the probes to IgL mRNA and IgK mRNA in the cytoplasm of the cells in the sample, to determine numbers of IgL-expressing cells and IgK-expressing cells.

11. The method of claim 1, wherein the one or more probes comprise probes that bind to a plurality of target regions in the IgL or IgK mRNA.

12. The method of claim 1, wherein the binding of the probes to IgL mRNA or IgK mRNA is detected using one or more labels that are directly or indirectly bound to the polynucleotide probes.

13. The method of claim 1, wherein the binding of the probes to IgL mRNA or IgK mRNA is detected using branched nucleic acid signal amplification, and the probes are branched DNA probes.

14. (canceled)

15. The method of claim 13, comprising contacting the sample with a plurality of probes that comprises one or more label extender probes that bind to one or more target regions in the IgL mRNA or IgK mRNA; hybridizing one or more pre-amplifier probes to the one or more label extender probes; hybridizing one or more amplifier probes to the pre-amplifier probes; and hybridizing one or more label probes to the one or more amplifier probes.

16. The method of claim 15, wherein the label probes are conjugated to an enzyme, and binding of the probe is detected using a chromogen substrate with the enzyme, or the label probes are conjugated to a fluorophore, and binding of the probe is detected by observation of emissions from the fluorophore after illumination suitable to excite the fluorophore.

17. (canceled)

18. The method of claim 1, further comprising:
contacting a sample comprising tissue from the tumor with one or more polynucleotide probes that bind specifically to one or more mRNAs encoding a housekeeping gene (HKG) in situ;
detecting binding of the one or more probes to one or more HKG mRNAs, and selecting for further analysis a sample in which binding of the one or more probes to the one or more HKG mRNAs are detected, or rejecting a sample in which binding of the one or more probes to the one or more HKG mRNAs are not detected.

19. (canceled)

20. (canceled)

21. (canceled)

22. (canceled)

23. (canceled)

24. (canceled)

25. (canceled)

26. The method of claim 1, wherein the cells of the sample were removed, at least in part, from a lymph node.

27. The method of claim 26, wherein a sample identified as not being associated with MM, HL, or NHL is classified as being from a normal lymph node or a reactive lymph node based on one or more morphological features.

28. The method of claim 27, wherein classification of a normal lymph node is made, at least in part, based on one or more of: a moderate expression of IgK/IgL within non-clonal lymphocytes of the lymphoid follicles; high expression of IgK/IgL within non-clonal plasma cells.

29. The method of claim 28, wherein moderate expression of IgK/IgL is indicated by detection of up to 20 IgK/IgL mRNAs per lymphocyte.

30. (canceled)

31. The method of claim 28, wherein high expression of IgK/IgL is indicated by detection of 100 or more IgK/IgL mRNAs per plasma cell.

32. The method of claim 27, wherein classification of a reactive lymph node is made, at least in part, based on one or more of:
the presence of greater than a threshold number of lymphoid follicles showing a non-clonal population of IgK/IgL expressing lymphocytes; the presence of less than a threshold number of the lymphoid follicles showing a clonal population of IgK/IgL expressing lymphocytes;
the presence of greater than a threshold number of non-clonal plasma cells per lymphoid follicle; or
absence of clonal effacement within lymphoid follicles.

33. The method of claim 32, wherein the threshold number of lymphoid follicles showing a non-clonal population of IgK/IgL expressing lymphocytes is 70% of the lymphoid follicles.

34. (canceled)

35. The method of claim 32, wherein the threshold number of the lymphoid follicles showing a clonal population of IgK/IgL expressing lymphocytes is 30% of the lymphoid follicles.

36. (canceled)

37. The method of claim 32, wherein the threshold number of non-clonal plasma cells per lymphoid follicle is 3 non-clonal plasma cells per lymphoid follicle.

38. (canceled)

39. The method of claim 1, wherein a sample identified as being associated with MM is identified, at least in part, based on one or more morphological features.

40. The method of claim 39, wherein identification of the sample as being associated with MM is made, at least in part, based on high expression of IgK/IgL within a clonal population of plasma cells.

41. The method of claim 40, wherein high expression of IgK/IgL is indicated by detection of 100 or more IgK/IgL mRNAs per plasma cell.

42. The method of claim 1, wherein a sample identified as being associated with NHL is identified, at least in part, based on one or more morphological features.

43. The method of claim 42, wherein identification of the sample as being associated with NHL is made, at least in part, based on one or more of:
moderate expression of IgK/IgL within a clonal expansion of lymphocytes within lymphoid follicles;
more than half of the lymphoid follicles showing lymphocytes in which the ratio of IgL-expressing B-lymphocytes to IgK-expressing B-lymphocytes, or ratio of IgK-expressing B-lymphocytes to IgL-expressing B-lymphocytes, is above the threshold;
presence of clonal effacement within lymphoid follicles; or
presence of less than a threshold number of plasma cells per lymphoid follicle.

44. The method of claim 43, wherein moderate expression of IgK/IgL is indicated by detection of up to 20 IgK/IgL mRNAs per lymphocyte.

45. (canceled)

46. (canceled)

47. (canceled)

48. The method of claim 45, wherein identification of the sample as being associated with NHL is made based on the presence of less than 7 plasma cells per lymphoid follicle.

49. (canceled)

50. (canceled)

51. (canceled)

52. (canceled)

53. (canceled)

54. A kit for performing the method of claim 1, wherein the kit comprises:
(A) one or more polynucleotide probes that bind specifically to IgK mRNA in situ comprising one or more label extender probes that are capable of binding to one or more target regions in the IgK mRNA; and
(B) one or more polynucleotide probes that bind specifically to IgL mRNA in situ.

55. The kit of claim 54, wherein the one or more polynucleotide probes that bind specifically to IgL mRNA in situ comprise one or more label extender probes that are capable of binding to one or more target regions in the IgL mRNA.

56. The kit of claim 54, wherein the kit further comprises one or more polynucleotide probes that bind specifically to mRNA encoding a housekeeping gene (HKG) in situ.


SEQUENCE LISTING (28 sequences)

SEQ ID NO: 1 - DNA, 819 bp, Homo sapiens
ggccagcttccctctcctcctcaccctcctcactcactgtgcagggtcctgggcccagtctgtgctgactcagccaccctcagcgtctgggacccccgggcagagggtcatcatctcttgttctggaagcagctccaacatcggaggtaatactgtaaactggtaccagcagctcccaggaagggcccccaaactcctcatccatagtaataatcagcggccctcaggggtccctgaccgattctctggctccaagtctggcacctcagcctccctggccatcagtgggctccagtctgaggatgaggctgattattactgtgcagcatgggatgacagcctgaatggtcggtatgtcttcggaactgggaccaaggtcaccgtcctaggtcagcccaaggccaaccccactgtcactctgttcccgccctcctctgaggagctccaagccaacaaggccacactagtgtgtctgatcagtgacttctacccgggagctgtgacagtggcctggaaggcagatggcagccccgtcaaggcgggagtggagaccaccaaaccctccaaacagagcaacaacaagtacgcggccagcagctacctgagcctgacgcccgagcagtggaagtcccacagaagctacagctgccaggtcacgcatgaagggagcaccgtggagaagacagtggcccctacagaatgttcataggttcccaactctaaccccacccacgggagcctggagctgcaggatcccaggggaggggtctctctccccatcccaagtcatccagcccttctccctgcactcatgaaacc

SEQ ID NO: 2 - DNA, 969 bp, Homo sapiens
ggggagtcagtcccagtcaggacacagcatggacatgagggtccccgctcagctcctggggctcctgctgctctggctcccaggtgccagatgtgacatccagttgacccagtctccatccttcctgtctgcagctgtgggagacagagtcagcatcacttgccgggccagtcaggacatcagcaaatatttagcctggtatcaacataaaattgggaaagcccctaaactcctgatctatggtgcatccactttgcaaagtggggtcccatcaagattcagtggcagtgggtctgggacagaattcactctcacaatcaacagcctgcagcctgaggatctcgcaacttattactgtcaacaacttaataattaccccctcactttcggcggggggaccaaggtggagatcatacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttagagggagaagtgcccccacctgctcctcagttccagcctgaccccctcccatcctttggcctctgaccctttttccacaggggacctacccctattgcggtcctccagctcatctttcacctcacccccctcctcctccttggctttaattatgctaatgttggaggagaatgaataaataaagtgaatctttgcacctgcaaaaaaaaaaaaaaaaaaaaaaaaaaaaaa

SEQ ID NO: 3 - DNA, 21 bp, Artificial Sequence
  synthetically generated Probe sequences
tgaagacagatggtgcagcca

SEQ ID NO: 4 - DNA, 20 bp, Artificial Sequence
  synthetically generated Probe sequences
ctcatcagatggcgggaaga

SEQ ID NO: 5 - DNA, 23 bp, Artificial Sequence
  synthetically generated Probe sequences
gaggcagttccagatttcaactg

SEQ ID NO: 6 - DNA, 23 bp, Artificial Sequence
  synthetically generated Probe sequences
agttattcagcaggcacacaaca

SEQ ID NO: 7 - DNA, 22 bp, Artificial Sequence
  synthetically generated Probe sequences
actttggcctctctgggataga

SEQ ID NO: 8 - DNA, 21 bp, Artificial Sequence
  synthetically generated Probe sequences
gcgttatccaccttccactgt

SEQ ID NO: 9 - DNA, 20 bp, Artificial Sequence
  synthetically generated Probe sequences
gggagttacccgattggagg

SEQ ID NO: 10 - DNA, 23 bp, Artificial Sequence
  synthetically generated Probe sequences
gtcctgctctgtgacactctcct

SEQ ID NO: 11 - DNA, 19 bp, Artificial Sequence
  synthetically generated Probe sequences
gcgtcagggtgctgctgag

SEQ ID NO: 12 - DNA, 23 bp, Artificial Sequence
  synthetically generated Probe sequences
tttctcgtagtctgctttgctca

SEQ ID NO: 13 - DNA, 20 bp, Artificial Sequence
  synthetically generated Probe sequences
tcgcaggcgtagactttgtg

SEQ ID NO: 14 - DNA, 19 bp, Artificial Sequence
  synthetically generated Probe sequences
caggccctgatgggtgact

SEQ ID NO: 15 - DNA, 21 bp, Artificial Sequence
  synthetically generated Probe sequences
gcgggaacagagtgacagtgg

SEQ ID NO: 16 - DNA, 21 bp, Artificial Sequence
  synthetically generated Probe sequences
cttggagctcctcagaggagg

SEQ ID NO: 17 - DNA, 22 bp, Artificial Sequence
  synthetically generated Probe sequences
cacactagtgtggccttgttgg

SEQ ID NO: 18 - DNA, 23 bp, Artificial Sequence
  synthetically generated Probe sequences
ccgggtagaagtcactgatcaga

SEQ ID NO: 19 - DNA, 20 bp, Artificial Sequence
  synthetically generated Probe sequences
ccaggccactgtcacagctc

SEQ ID NO: 20 - DNA, 18 bp, Artificial Sequence
  synthetically generated Probe sequences
ggggctgccatctgcctt

SEQ ID NO: 21 - DNA, 19 bp, Artificial Sequence
  synthetically generated Probe sequences
tctccactcccgccttgac

SEQ ID NO: 22 - DNA, 20 bp, Artificial Sequence
  synthetically generated Probe sequences
ctgtttggagggtttggtgg

SEQ ID NO: 23 - DNA, 22 bp, Artificial Sequence
  synthetically generated Probe sequences
cctggcagctgtagcttctgtg

SEQ ID NO: 24 - DNA, 19 bp, Artificial Sequence
  synthetically generated Probe sequences
gtgctcccttcatgcgtga

SEQ ID NO: 25 - DNA, 20 bp, Artificial Sequence
  synthetically generated Probe sequences
gggccactgtcttctccacg

SEQ ID NO: 26 - DNA, 25 bp, Artificial Sequence
  synthetically generated Probe sequences
ttgggaacctatgaacattctgtag

SEQ ID NO: 27 - DNA, 19 bp, Artificial Sequence
  synthetically generated Probe sequences
cccgtgggtggggttagag

SEQ ID NO: 28 - DNA, 19 bp, Artificial Sequence
  synthetically generated Probe sequences
gatcctgcagctccaggct